SEQUENCE LISTING (17 sequences)

SEQ ID NO: 1 - DNA, 23 bp, Artificial Sequence
  m157 forward PCR primer
tcaacttccgacgcaaaagaaat

SEQ ID NO: 2 - DNA, 21 bp, Artificial Sequence
  m157 reverse PCR primer
accgtcgattcgtcagtaacg

SEQ ID NO: 3 - DNA, 35 bp, Artificial Sequence
  B2m forward wild type PCR primer
acggtgcagactgagctctgttttcatctgtcttc

SEQ ID NO: 4 - DNA, 35 bp, Artificial Sequence
  B2m reverse PCR Primer
caggcgtatgtatcagtctcagtgggggtgaattc

SEQ ID NO: 5 - DNA, 30 bp, Artificial Sequence
  B2m forward mutant PCR Primer
tcaggatgatctggacgaagagcatcaggg

SEQ ID NO: 6 - DNA, 35 bp, Artificial Sequence; identical to SEQ ID NO: 4
  B2m reverse PCR Primer

SEQ ID NO: 7 - DNA, 20 bp, Artificial Sequence
  Act-mOVA forward PCT Primer
cttccaggattcggagacag

SEQ ID NO: 8 - DNA, 20 bp, Artificial Sequence
  Act-mOVA reverse PCR Primer
tctgaaaggcattgcttgtg

SEQ ID NO: 9 - DNA, 24 bp, Artificial Sequence
  Tgfbr2 forward PCT Primer
tatggactggctgcttttgtattc

SEQ ID NO: 10 - DNA, 24 bp, Artificial Sequence
  Tgfbr2 reverse PCR Primer
tggggatagaggtagaaagacata

SEQ ID NO: 11 - DNA, 30 bp, Artificial Sequence
  Hobit forward PCR Primer
tgcctccttttgacccatgacatccaaatt

SEQ ID NO: 12 - DNA, 30 bp, Artificial Sequence
  Hobit reverse PCR Primer
ctctttctggctcatttcccatctcctctc

SEQ ID NO: 13 - DNA, 20 bp, Artificial Sequence
  Hobit mutant PCR Primer
atcagttcgcttctcgcttc

SEQ ID NO: 14 - DNA, 23 bp, Artificial Sequence
  Lair1 forward wild type PCR Primer
ggtttgaggacagacagatgacc

SEQ ID NO: 15 - DNA, 22 bp, Artificial Sequence
  Lair1 reverse PCR Primer
tttatgaggaggacgactgagc

SEQ ID NO: 16 - DNA, 19 bp, Artificial Sequence
  Lair1 forward mutant PCR Primer
ccaggtgcacatgatcagc

SEQ ID NO: 17 - DNA, 22 bp, Artificial Sequence
  Lair1 reverse PCR Primer
tttatgaggaggacgactgagc

(19) United States
(12) Patent Application Publication
(10) Pub. No.: US 2024/0287194 A1
(43) Pub. Date: Aug. 29, 2024

(54) Targeting Extracellular Matrix Proteins to Regulate NK Cell Function in Peripheral Tissues

(71) Applicant: The General Hospital Corporation, Boston, MA (US)

(72) Inventor: Shadmehr Demehri, Charlestown, MA (US)

(21) Appl. No.: 18/289,836

(22) PCT Filed: May 13, 2022
(86) PCT No.: PCT/US2022/072319

§ 371 Date: Nov. 7, 2023

Related U.S. Application Data
(60) Provisional application No. 63/275,517, filed on Nov. 4, 2021.
(60) Provisional application No. 63/188,122, filed on May 13, 2021.

(51) Int. Cl. C07K 16/28; A61K 31/192; A61K 31/4178; A61K 39/00; A61P 35/00

(52) U.S. Cl. CPC C07K 16/2848; A61K 31/192; A61K 31/4178; A61P 35/00; C07K 16/2803; C07K 16/2842; A61K 2039/505; C07K 2317/76

(57) ABSTRACT

Elucidating the mechanism that underlies tissue-specific NK cell responses will have major implications in cancer immunology, transplant biology and virology. Provided herein are compositions and methods for of treating a subject who has a solid tumor or who has a viral infection, or promoting cytotoxicity of natural killer (NK) cells in a subject, using (i) blocking antibodies to ECM components and/or (ii) inhibitors of collagen deposition or production.

### CLAIM OF PRIORITY

This application claims the benefit of U.S. Provisional Application Ser. No. 63/188,122, filed on May 13, 2021, and 63/275,517, filed on Nov. 4, 2021. The entire contents of the foregoing are incorporated herein by reference.

### TECHNICAL FIELD

Provided herein are compositions and methods for of treating a subject who has a solid tumor or who has a viral infection, or promoting cytotoxicity of natural killer (NK) cells in a subject, using (i) blocking antibodies to ECM components and/or (ii) inhibitors of collagen deposition or production.

### BACKGROUND

It has long been appreciated that downregulation of Major Histocompatibility Complex Class I (MHC-I) is a primary mechanism for immune evasion utilized by cancer cells (1). In the era of cancer immunotherapeutics, MHC-I downregulation has emerged as a major resistance mechanism against immunotherapy as malignant cells escape from CD8⁺ T cell detection and elimination (1). However, the pervasive selection of this immune evasion strategy by cancer cells is perplexing in that MHC-I downregulation is expected to render cells vulnerable to Natural killer (NK) cell killing (2-4). The dilemma of NK cells' role in controlling cells that fail to express self MHC-I in vivo was originally encountered in the transplantation field. In the context of hybrid resistance, parental skin grafts are accepted, but bone marrow (BM) cells are rejected by F1 hybrids in an NK cell-dependent manner (5, 6). Subsequent studies have shown that NK cells in wild-type (WT) syngeneic recipients reject donor MHC-I-deficient BM cells, while MHC-I-deficient solid organ transplants are not rejected (7, 8). Elucidating the mechanism that underlies tissue-specific NK cell responses will have major implications in cancer immunology, transplant biology and virology.

NK cells belong to the innate lymphoid cell (ILC) family that reside in most tissues and form a swift acting innate barrier against viral infections and cells undergoing malignant transformation (9). Conventional NK (cNK) cells, defined by expression of NK1.1, NKp46 and CD49b in mice, are found in circulation, secondary lymphoid organs and most other tissues (9). Another member of the ILC family that is closely related in phenotype and function is the tissue-resident NK (trNK) or ILC1 cell that lacks CD49b but expresses CD49a (9). These cells are a non-migrating, NK cell-like population (10) that are thought to develop from two distinct pathways: 1) differentiation from the innate lymphoid cell progenitor (ILCP) (11) or 2) differentiation from cNK cells in the presence of TGFβ (12, 13) in a non-mutually exclusive manner.

### SUMMARY

Provided herein are methods for treating a subject who has a solid tumor and/or a viral infection. The methods comprise administering to the subject a therapeutically effective amount of (i) blocking antibodies to ECM components and/or (ii) inhibitors of collagen deposition or production.

Also provided are methods for promoting cytotoxicity of natural killer (NK) cells in a subject. The methods comprise administering to the subject a therapeutically effective amount of (i) blocking antibodies to ECM components and/or (ii) inhibitors of collagen deposition or production.

Also provided are compositions comprising (i) blocking antibodies to ECM components and/or (ii) inhibitors of collagen deposition or production, for use in a method of treating a subject who has a solid tumor, a viral infection, and/or for promoting cytotoxicity of natural killer (NK) cells in a subject.

In some embodiments, the blocking antibodies to ECM components bind to LAIR1/2; Cd29 (integrin R); CD49A; GPR561; integrin αV or integrin β3.

In some embodiments, the inhibitor of collagen deposition or production is losartan or 3,4-dihydroxybenzoic acid (DHB).

In some embodiments, the subject has breast, prostate, pancreatic, brain, hepatic, lung, kidney, skin, or colon cancer. In some embodiments, the skin cancer is melanoma.

In some embodiments, the subject has a viral infection selected from a respiratory virus (optionally influenza viruses (A and B), H5N1 and H7N9 avian influenza A viruses, parainfluenza viruses 1 through 4, adenoviruses, respiratory syncytial virus A and B and human metapneumovirus, rhinoviruses, or coronaviruses); a gastrointestinal virus (optionally rotavirus, norovirus, astrovirus, adenovirus 40 and 41, or coronavirus-like agents, or enteroviruses, optionally coxsackieviruses and echoviruses); hepatitis A, B, C, D, and E viruses; arboviruses, arenaviruses, and filoviruses; and viruses that infect the skin or mucosal membranes (optionally herpes simplex viruses (HSV), papillomavirus, polyomavirus, and poxviruses).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-G. Presence of NK cell activating m157 ligand and loss of inhibitory B2m are insufficient to induce skin graft rejection in syngeneic recipients.
(A) Schematic diagram of ear skin transplantation from B6 albino WT, B2m^{-/-}, m157^{tg} and m157^{tg}, B2m^{-/-} donor mice to B6 Ncr1^{iCre}, ROSA^{mT-mG} recipients.
(B) Representative images of skin grafts at day 20 post-transplant.
(C) Quantification of graft rejection grades assessed at day 20 post-transplant of B6 albino WT, B2m^{-/-}, m157^{tg} and m157^{tg}, B2m^{-/-} skin grafts transplanted onto B6 Ncr1^{iCre} ROSA^{mT-mG} recipients.
(D) The percentage change in skin graft size from day 10 to day 20 post-transplant.
(E) Representative IF images of NKp46-GFP⁺ (green), CD4⁺ (purple) and CD8⁺ (red) cells in skin grafts at day 20 post-transplant. Dotted lines indicate the epidermal basement membrane.
(F) Quantification of NKp46-GFP⁺ NK, CD4⁺ T and CD8⁺ T cells in the skin and epidermis of skin grafts at day 20 post-transplant. (B-F) n=9-12 mice per group from nine independent experiments.
(G) Time-course of skin graft maintenance from day 10 to day 20 post-transplant comparing m157^{tg}, m157^{tg}, B2m^{-/-}, mOVA^{tg} and mOVA^{tg}, B2m^{-/-} skin grafts. m157^{tg} and m157^{tg}, B2m^{-/-} images from the same experiment as (B). mOVA^{tg} and mOVA^{tg}, B2m^{-/-} n=2-5 from one to two independent experiments.
(C) Fisher's exact test. ns: not significant, * p<0.05, *** p<0.001 compared with WT group. (D and F) Graphs show mean±SD. Mann-Whitney (test, ns: not significant, * p<0.05, ** p<0.01, *** p<0.001. (B and G) Scale bars=1 cm, (E) scale bar=100 μm.

FIGS. 2A-G. NK cells fail to induce skin graft rejection upon cytokine stimulation.
(A) Schematic of Poly(I:C)+mIL-12/mIL-15 and mIL-12/mIL-15/mIL-18 injections.
(B-E) Proportion of total NK cells (B) CD11b⁺CD27⁻ mature NK cells (C), PD1⁺NK cells (D) and cNK (CD49b⁺), dpNK (CD49b⁺CD49a⁺) and trNK (CD49a⁺) cells (E) in m157^{tg}, B2m^{-/-} skin grafts at day 21 post-transplant in B6 mice treated with PBS (control) versus IL-12/IL-15/IL-18 cytokines as depicted in (A) (n=4-5 per group).
(F) Enumeration of cNK, dpNK and trNK populations in the skin by combining flow cytometry frequency data with NKp46-GFP⁺ cell counts from FIG. 1. n=9-10 mice per group from nine independent experiments.
(G) Ly49H expression by cNK, dpNK and trNK cells from m157^{tg}, B2m^{-/-} and cNK cells from WT skin grafts at day 20 post-transplant compared with cNK cells from the liver. Note that WT skin grafts contain Ly49H⁺ and Ly49H⁻ cNK cells while all NK subsets in m157^{tg}, B2m^{-/-} skin grafts express low levels of Ly49H. Average±SD of mean fluorescent intensity (MFI) of Ly49H on NK cell subsets in m157^{tg}, B2m″ skin grafts and Ly49H⁻ cNK cells in WT Skin grafts at day 20 post-transplant are compared with Ly49H⁻ cNK cells in the liver of the recipient mice. n=4-10 mice per group from three independent experiments. Graphs shown mean±SD. Mann-Whitney U test, ns: not significant, * p<0.05, ** p<0.01, *** p<0.001.

FIG. 3| Presence of activating m157 and loss of self-MHC induces robust skin graft rejection in allogeneic recipients. Quantification of NKp46-GFP⁺ NK, CD4⁺ T and CD8⁺ T cells in the skin grafts at days 3, 5, 7 and 10 post-transplant. Red arrow highlights NK cells infiltrating the skin prior to T cells. n=3-9 mice per group from one to three independent experiments.

FIGS. 4A-M. Dermal ECM proteins modulate cNK cell effector function in vitro.
(A, B) Quantification of CD107a (A) and IFNγ (B) by Ly49H⁺ splenic cNK cells after 7 hours co-culture with no MEF, WT-MEF or m157-MEF in the presence of IL-12 and IL-15. n=5 per group.
(C) Representative dot plots of CD107a by splenic cNK cells co-cultured with WT-MEF or m157-MEF. Representative histograms of Ly49H expression by Ly49H CD107a⁺ NK cells following co-culture with m157-MEF and WT-MEF. Histograms of Ly49H⁺CD107a⁻ cNK cells (black) are shown as controls. Numbers on the dot plots represent the percent cells within each gate.
(D-G) Quantification of CD107a (D, E) and IFNγ (F, G) expression by Ly49H⁺ splenic cNK cells co-cultured with different ECM proteins and m157-MEF at 9 hours (D, F) and 24 hours (E, G). Percentages are normalized based on the “No ECM” group. n=7 per group from two independent experiments.
(H, I) Comparison of CD 107a (H) and IFNγ (I) by Ly49H⁺ splenic cNK cells from WT and Lair1^{-/-} mice in the presence of m157-MEF and collagen I. Percentages are normalized based on “No ECM” group in WT (n=7) and Lair1^{-/-} (n=12) experiments.
(J-M) Measurement of CCL2 (J), CXCL10 (K), CCL5 (L) and CXCL9 (M) in culture media of splenic cNK cells co-cultured with m157-MEF in the presence of no ECM, collagen I, III or elastin at 12 hours (CXCL10, CCL5) and 24 hours (CCL2, CXCL9). n=8 per group from two independent experiments.
(A, B, D-M) Graphs show mean±SD. Mann-Whitney U test, ns: not significant, * p<0.05, ** p<0.01, *** p<0.001 compared with no ECM group.

FIGS. 5A-I. Collagens and elastin modulate cytotoxicity and inflammation-associated signaling pathways in NK cells.
(A) GSEA enrichment plots of PI3K-AKT-mTOR, TNFα-NF□B, IL-6-JACK-STAT3 and IL-2-STAT5 signaling in CD49b⁺ cNK cells in the donor skin compared with the circulation. The enrichment scores (ES) and P values are indicated in each plot.
(B) Quantification of phospho(p)NFκB (p65) in splenic cNK cells at 30 and 60 minutes post-coculture with m157-MEF in the presence of different ECM proteins. n=4 per group, data representative of two independent experiments.
(C-G) Quantification of phospho(p)PLCγ1⁺ (C), pERK1/2⁺ (D), pNFκB (p65)⁺ (E), pSTAT3⁺ (F) and pSTAT5⁺ (G) in splenic cNK cells at 2- and 5-minutes post-stimulation with H₂O₂ in the presence of different ECM proteins. n=8 per group from two independent experiments. Representative flow cytometry histograms comparing the phosphorylation of key signaling proteins at 2- and 5-minutes post-stimulation with H₂O₂ in response to no ECM (first set of three bars), collagen I (second set of three bars), collagen III (third set of three bars) and elastin (fourth set of three bars). Black dotted line corresponds to the gating based on the “no ECM-0 min” histogram for each signaling protein.
(H, I) Comparison of pERK1/2 (H) and pNFκB (p65) (I) in splenic NK cells from WT and Lair1^{-/-} mice in response to 2 minutes H₂O₂ stimulation in the presence of collagen I. Percentages are normalized based on “no ECM” group in WT and Lair1^{-/-} experiments. n=4 per group, data is representative of two independent experiments.
(B-I) Graphs show mean±SD. Mann-Whitney U test, ns: not significant, * p<0.05, ** p<0.01, *** p<0.001. (C-G) each time point is compared with the corresponding time point in no ECM group.

FIGS. 6A-B. B16 melanoma infiltrating NK cells are found to interact with collagen within the tumor stroma.
(A) Schematic diagram of WT and B2m^{-/-} B16 melanoma cell inoculation and anti-NK1.1 antibody treatment for the subcutaneous (S.C.) melanoma model. Note: Losartan, DHB or PBS (carrier) treatments were given to mice receiving B2m^{-/-} B16 melanoma cells.
(B) WT B16 melanoma subcutaneous (S.C.) growth rate over time in WT mice treated with control IgG (n=17 for tumor growth) or anti-NK1.1 antibody (n=19 for tumor growth). Two-way ANOVA, ns: not significant.

FIGS. 7A-L. Collagen deposition blocks NK cells from eliminating B2m-deficient melanoma cells in the skin.
(A, B) B2m/B16 melanoma S.C. growth over time (A) and terminal weight
(B) in WT mice treated with control IgG (n=17 for (A) and 13 for (B)) or anti-NK1.1 antibody (n=19 for (A) and n=13 for (B)).
(C) Representative images of NKp46-GFP⁺ NK cells (white arrows) immunofluorescence and collagen stain in adjacent sections of a B2m^{-/-} B16 melanoma at day 3 post-S.C. injection in Ncr1^{iCre}, ROSA^{mT-mG} mice.
(D-G) B2m/B16 melanoma S.C. growth over time (D, F) and terminal weight (E, G) in WT mice treated with Losartan (D, E) or DHB (F, G) plus control IgG (n=28 (D), 20 (F), 27 (E) and 20 (G)) or anti-NK1.1 antibody (n=26 (D), 20 (F), 25 (E) and 18 (G)).
(H, I) Representative images (H) and quantification (I) of NKp46-GFP⁺ NK cells in B2m^{-/-} B16 melanoma at day 3 post-S.C. injection in PBS (n=10), Losartan (n=7) and DHB (n=9) treated Ncr1^{iCre},ROSA^{mT-mG} mice.
(J-L) Quantification of Granzyme B (J), IFNγ production (K) and PD-1 surface expression (L) on NK cells isolated from B2m^{-/-} B16 melanoma at day 19-21 post-S.C. injection in PBS (n=13 (J, L), 8 (K)), Losartan (n=21 (J), 22 (K, L)) and DHB (n=20 (J, K, L)) treated mice.
(A, D, F) Two-way ANOVA, (B, E, G, I-L) Graphs show mean±SD, Mann-Whitney U test. ns: not significant, * p<0.05, ** p<0.01, *** p<0.001. (C and H) Scale bars=100 μm.

FIGS. 8A-L. Inhibition of collagen deposition leads to NK cell-mediated control of B2m^{-/-} melanoma in the skin.
(A, B) B2m/B16 melanoma S.C. growth over time (A) and terminal weight (B) in WT mice treated with PBS (gray; n=28 (A), 24 (B)), losartan (orange; n=28 (A), 27 (B)) or DHB (purple; n=20 (A and B)). Note that all animals received control IgG and the data are also shown in FIG. 6, A, D and F (IgG groups).
(C) Kaplan-Meier survival plot for WT mice treated with PBS (gray; n=28), Losartan (orange; n=28) and DHB (purple; n=20) following subcutaneous injection of B2m^{-/-} B16 melanoma cells. Note that all animals received control IgG and tumor growth from these mice is shown in (A). Mice were considered moribund when tumor volume reached 1 cm³.
(D) B2m/B16 melanoma S.C. growth over time in WT mice treated with anti-NK1.1 antibody plus PBS (magenta; n=19), losartan (green; n=26) or DHB (blue; n=20). Note that the data are also shown in FIG. 6, A, D and F (αNK1.1 groups).
(E, F) Representative images (E) and quantification (F) of CD3+ T cells in B2m^{-/-} B16 melanoma at day 3 post S.C. injection in PBS (n=9), Losartan (n=9) and DHB (n=9) treated Ncr1^{iCre}, Rosa^{mT-mG} mice. (E) Scale bar=100 μm.
(G) Proportion of CD3+CD4*Foxp3⁺ regulatory T cells (Tregs) in B2m^{-/-} B16 melanoma from WT mice treated with PBS (n=17), Losartan (n=29) and DHB (n=20) examined by flow cytometry at the endpoint. (H-J) Proportion of CD11b⁺ (H), F4/80+(I) and Arginase1⁺ (J) cells in B2m^{-/-} B16 melanoma from WT mice treated with PBS (n=9), Losartan (n=7) and DHB (n=9) examined by immunofluorescence staining at the endpoint.
(K) B2m/B16 melanoma S.C. terminal weight in WT mice treated with collagenase and hyaluronidase plus control IgG (n=24) or anti-NK1.1 antibody (n=18).
(L) Scoring S.C. B2m^{-/-} B16 melanoma metastasis to the draining lymph nodes in WT mice treated with PBS, losartan or DHB and received control IgG versus anti-NK1.1 antibody. PBS: IgG (n=8) and anti-NK1.1 (n=8); losartan: IgG (n=19) and anti-NK1.1 (n=23); DHB: IgG (n=15) and anti-NK1.1 (n=18), Fisher's exact test.
(A, D) Two-way ANOVA, ns: not significant, * p<0.05, ** p<0.01, *** p<0.001. (C) Log-rank (Mantel-Cox) test. (B, F-K) graphs show mean±SD. Mann-Whitney U test, ns: not significant, * p<0.05, ** p<0.01, *** p<0.001.

FIGS. 9A-C. Impact of NK cells on WT and B2m^{-/-} melanoma lung metastases.
(A) Schematic diagram of WT and B2m^{-/-} B16 melanoma cell inoculation and anti-NK1.1 antibody treatment for the metastatic (I.V.) melanoma model.
(B) Quantification of WT B16 melanoma metastatic foci in the lung following tail vein I.V. injection and treatment with control IgG versus anti-NK1.1 antibody. n=10 mice per group.
(C) Quantification of B2m/B16 melanoma metastatic foci in the lung following tail vein I.V. injection and treatment with control IgG versus anti-NK1.1 antibody. n=10-15 mice per group.
(B, C) Graphs show mean±SD. Mann-Whitney U test, ns: not significant, p<0.01, *** p<0.001, compared with IgG-treated group.

FIG. 10. B2M gene alterations in solid versus blood cancers in humans.

B2M gene alteration frequency in various solid cancers and leukemias from the TCGA pan-cancer dataset and other public cancer databases from eBioPortal.

### DETAILED DESCRIPTION

Full NK cell activation against a target cell is accomplished by the integration of multiple inhibitory and activation signals, a process that limits indiscriminate killing of healthy host cells. Self MHC-I molecules function as the dominant inhibitory signal by binding NK cell-expressed Ly49 receptors in mice, or killer inhibitory receptors (KIR) in humans, to prevent degranulation and cytokine release. Multiple activation signals, together with downregulation or loss of inhibitory signals, are required to enable complete NK cell cytotoxicity (3, 14, 15). Following their licensing to activate their cytotoxic program (16-18), NK cells destroy the target cells by production and release of granzymes and perforin in the immunological synapse (19). Fas-FasL and TRAIL are also used by NK cells for target cell elimination (20).

Although in vitro models of NK cell activation have provided insights into signaling requirements that initiate NK cell killing of target cells, the conditions in vivo are much more complex in that non-MHC ligands and potentially unknown inhibitory and activation pathways of varying strengths, acting together with cytokines, set the NK cell activation threshold (21). Most in vivo NK cell activation models have focused on NKG2D and other activating ligands and cytokines that also activate CD8⁺ T cells (22), complicating interpretation of NK versus CD8⁺ T cell effects. To investigate the mechanism of NK cell activation in peripheral tissues, we utilized the NK cell-specific m157-Ly49H activation axis in a skin transplantation model. m157 is murine cytomegalovirus (MCMV)-encoded glycoprotein expressed on the surface of infected cells. This MHC-I-like molecule serves as an NK cell-specific activation signal, directing the targeted killing of MCMV infected cells through recognition by the Ly49H receptor, which is only present on the surface of NK cells on the C57BL/6 (B6) background (23). As a result, B6 mice are resistant to MCMV infection, while BALB/c and other strains are susceptible (24). Thus, the m157-Ly49H axis can be used to specifically study NK cell activation and its downstream function in vivo.

Herein, we demonstrate that upon exit from the circulation into the skin graft in response to m157, NK cells are exposed to extracellular matrix (ECM) proteins, which can block NK cell cytotoxicity while promoting its helper function. NK cell activation induced by the combination of m157 and missing self MHC-I (25) resulted in skin graft rejection in an NK and T cell-dependent manner. RNA-Seq analysis revealed significant downregulation in cytotoxic mediators together with upregulation in inflammatory cytokines and chemokines, when comparing cNK cells that entered the skin with circulating cNK cells. We found that in vitro exposure to ECM components, collagens and elastin, transformed the function and signaling within cNK cells. Furthermore, control of circulating melanoma cells occurred in an NK cell-dependent manner, yet the growth of these same melanoma cells subcutaneously was not impacted by NK cells embedded in ECM. Importantly, blocking collagen deposition in subcutaneous melanomas led to an NK cell-mediated tumor suppression. Human solid cancers, but not leukemias, could afford to downregulate MHC-I to escape CD8⁺ T cell-mediated elimination, which suggested that NK cells within peripheral tissues, unlike those in circulation, lacked a direct cytotoxic function. This study reveals a fundamental aspect of NK cell biology, which governs the interplay of NK cells with cancer, organ transplantation and viral infection.

NK cell killing of virally infected or malignant cells is a vital aspect of innate immunity. Yet, as noted above the mechanisms behind the inability of NK cells to directly eliminate NK cell-sensitive targets in peripheral tissues have remained undetermined. We show here, using skin transplantation and cancer models, that once NK cells exit the circulation and enter the target tissue, they experience a profound change in their function (FIG. 9). The present data strongly suggest that the interplay between several ECM proteins and NK cell receptors play a cooperative role in this functional shift immediately upon entry of cNK cells into the skin. NK cell-ECM protein interactions suppress NK cell's direct cytotoxicity and instead promote its helper function to recruit and activate adaptive immunity. Although this regulation of NK cell cytotoxicity may limit nonspecific tissue damage in the context of localized viral infection in peripheral tissues, it allows the outgrowth of cancer cells that have managed to evade cytotoxic T cell immunity.

Large numbers of NK cells are recruited into m157-expressing donor skin; however, NK cells are unable to directly reject the skin grafts even with the loss of MHC-I and the addition of NK cell-stimulating poly(I:C) and cytokines in a syngeneic model. This demonstrates that the MHC-I-independent inhibition of NK cell's cytotoxicity dominated over multiple strong activation signals tested. Skin graft rejection is accomplished using F1 recipients, which provides missing-self for NK cell activation while simultaneously preserving MHC-I expression on the donor cells. In this system, NK cells licensed by BALB/c MHC-I are fully activated against B6 MHC-I-expressing m157⁺ donor skin cells (i.e., missing-self) (25) while CD8⁺ T cells are able to contribute to the rejection as the MHC-I signaling axis remains operational. Graft rejection begins after day 12 post-transplant, which is approximately 7 days later than the initial accumulation of NK cells in the graft. The majority of rejection is completed by day 20 post-transplant, which is in agreement with the timing of an adaptive immune response. The timing of NK and T cell infiltration into the skin graft, the kinetics of graft rejection and the requirement for both NK and T cells in this process suggest that NK cells activate the adaptive immune response and T cells function as effectors to reject m157^{tg} donor skin grafts in F1 recipients.

NK cell activation has been associated with the production of high levels of IFNγ and TNFα in addition to CCL4, CCL5, XCL1, and GM-CSF (44-46). These cytokines and chemokines promote macrophage activation together with recruitment and activation of DCs and T cells at sites of inflammation (47-51). Interestingly, activated cNK cells entering the skin from circulation immediately upregulated the CXCR3 ligands, CXCL9 and CXCL10, in addition to CCL2 and CXCL5, chemokines that profoundly induce T cell recruitment. In contrast, CCL5 was downregulated and CCL4, XCL1, and GM-CSF expression were not altered in cNK cells as they exit the circulation. A transient increase in CCR7 is also observed, which may drive newly recruited cNK cells deeper into the skin, and subsequently the draining lymph nodes, where CCL21⁺ lymphatic endothelial cells reside. A concomitant reduction in the expression of T-bet and EOMES and an increase in ICOS may drive a helper cNK cell functional program. Furthermore, the cytotoxic effectors Gzma, Gzmb and Prf1 were either reduced or not changed in activated cNK cells as they exit the circulation, supporting observations of limited NK cell direct cytotoxicity in the skin. Without wishing to be bound by theory, although NK cells may directly activate CD8⁺ T cells in the skin graft, an indirect path through dendritic cells and/or CD4⁺ T cell activation likely plays an important role in the observed NK cell helper function, which will be further investigated in future studies (51-53). While direct killing of some target cells by NK cells may occur, the rejection of m157^{tg} skin graft is driven by CD8⁺ and CD4⁺ cells, which likely recognize m157 as foreign antigen expressed by the donor skin cells (54-56).

Previous studies have shown a role for TGFβ in impairing NK cell function in cancer models by promoting the conversion of cNK cells to trNK cells (12, 13, 37). As shown herein, the impairment in NK cell cytotoxicity is an immediate consequence of cNK cell exit from the circulation into a peripheral tissue microenvironment. In response to skin transplantation, CD49a CD49b trNK cells develop from cNK cells that have exited the circulation and populated the skin graft over time. Interestingly, cNK cells upregulated TGFβ receptor as soon as they exited circulation, which may promote their conversion into trNK cells in the TGFβ-rich environment of the skin graft. Although they may provide cytokine-mediated help to T cells later in the response, trNK cells are not seen in significant numbers until after day 14 post-transplant. In addition, blocking trNK cell development by deleting Tgfbr2 (12, 13) or Hobit transcription factor (38) does not result in NK cell cytotoxicity or graft rejection. These findings demonstrate that the switch in NK cell function from killer to helper is an immediate consequence of its exit from circulation and entry into the peripheral tissue microenvironment.

Cancers in peripheral tissues that are expected to be strong targets for NK cells have often failed to show objective responses (57-59). These results suggest that NK cells experience inhibition of their cytotoxicity likely mediated by collagens and elastin once they exit the circulation and enter the stroma. This may explain the distinct selection for loss of MHC-I by solid cancers but not leukemias in humans.

Described herein are methods for promoting cytotoxicity of NK cells in peripheral tissues by inhibiting the interaction of NK cells with components of the ECM. These methods are useful, e.g., in treating solid tumors and viral infections.

### Methods of Treatment

The present methods include administration of therapeutically effective amounts of blocking antibodies to ECM components and/or inhibitors of collagen deposition. In some embodiments, the administration is local administration, e.g., by injection or infusion into or near a tumor, or systemic administration, e.g., by intravenous injection or infusion.

### Cancer

The methods described herein include methods for the treatment of cancer. In some embodiments, the cancer is a solid tumor, e.g., breast, prostate, pancreatic, brain, hepatic, lung, kidney, skin, or colon cancer. Generally, the methods include administering a therapeutically effective amount of a treatment as described herein, to a subject who is in need of, or who has been determined to be in need of, such treatment.

As used in this context, to “treat” means to ameliorate at least one symptom of the disorder. For example, a treatment can result in a reduction in tumor size or growth rate. Administration of a therapeutically effective amount of a compound described herein for the treatment of a condition associated with abnormal apoptotic or differentiative processes will result in a reduction in tumor size or decreased growth rate, a reduction in risk or frequency of reoccurrence, a delay in reoccurrence, a reduction in metastasis, increased survival, and/or decreased morbidity and mortality, inter alia.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

In some embodiments, the cancer is not a hematopoietic neoplastic disorder. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof.

Methods for identifying and diagnosing subjects with cancer, e.g., with solid tumors, are known in the art.

### Viral Infection

The methods described herein include methods for the treatment of viral infections, to boost NK cytotoxicity in virally infected subjects. In some embodiments, the viral infection is an infection with a respiratory virus, e.g., influenza viruses (A and B), H5N1 and H₇N9 avian influenza A viruses, parainfluenza viruses 1 through 4, adenoviruses, respiratory syncytial virus A and B and human metapneumovirus, rhinoviruses, or coronaviruses; a gastrointestinal virus, e.g., rotavirus, norovirus, astrovirus, adenovirus 40 and 41, or coronavirus-like agents, as well as enteroviruses, which include coxsackieviruses and echoviruses; hepatitis A, B, C, D, and E viruses; arboviruses, arenaviruses, and filoviruses; viruses that infect the skin or mucosal membranes like herpes simplex viruses (HSV), papillomavirus, polyomavirus, and poxviruses. Topical/local or systemic administration can be used as appropriate. For example, to treat infections in the lungs, administration by inhalation or insufflation, e.g., of an aerosol or fine powder, can be used; for infections of skin or mucosal membranes, topical delivery method can apply. Systemic administration can also be used.

Generally, the methods include administering a therapeutically effective amount of a treatment as described herein, to a subject who is in need of, or who has been determined to be in need of, such treatment, e.g., who has been identified as having a viral infection as described herein. Methods for identifying and diagnosing subjects with viral infections are known in the art. In some embodiments, the treatment is administered systemically, e.g., parenterally or orally; in some embodiments, e.g., where a specific organ or tissue is infected, the treatment is administered more locally or directly to the affected tissues, e.g., topically, vaginally, rectally, orally, ocularly, or by inhalation.

### Blocking Antibodies to ECM Components

The present methods include the use of blocking antibodies to ECM components, including antibodies to LAIR 1/2; CD29 (integrin R); CD49A; GPR561 and integrin αV or β3. The methods include the administration of these antibodies (or combinations thereof, e.g., combination of lair1 plus CD29 (CD49b and CD49a) antibodies), or fusion proteins comprising the antibodies (or combinations thereof), that block ECM protein/receptor interaction. For example, binding and functional assays of NK cell activation and target killing in the presence of ECM proteins +/−antibody can be used to determine the efficacy of antibody blockade.

The term “antibody” as used herein refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments the antibody has effector function and can fix complement. In some embodiments, the antibody has reduced or no ability to bind an Fc receptor. For example, the antibody can be an isotype or subtype, fragment or other mutant, which does not support binding to an Fc receptor, e.g., it has a mutagenized or deleted Fc receptor binding region. Methods for making antibodies and fragments thereof are known in the art, see, e.g., Harlow et. al., editors, Antibodies: A Laboratory Manual (1988); Goding, Monoclonal Antibodies: Principles and Practice, (N.Y. Academic Press 1983); Howard and Kaser, Making and Using Antibodies: A Practical Handbook (CRC Press; 1st edition, Dec. 13, 2006); Kontermann and Dübel, Antibody Engineering Volume 1 (Springer Protocols) (Springer; 2nd ed., May 21, 2010); Lo, Antibody Engineering: Methods and Protocols (Methods in Molecular Biology) (Humana Press; Nov. 10, 2010); and Dübel, Handbook of Therapeutic Antibodies: Technologies, Emerging Developments and Approved Therapeutics, (Wiley-VCH; 1 edition Sep. 7, 2010).

Exemplary blocking antibodies are shown in the following table A.

TABLE A
Blocking antibodies

| ECM receptor | | | |
|---|---|---|---|
| (target) | Clone | Function | Company (Cat Nr.) |
| Lair1 | DX26 | Blocking (soluble)/ | BD Biosciences |
| | | agonist (coated) | (550810) |
| Lair1 | NKTA255 | Blocking (soluble)/ | Thermo Scientific |
| | | agonist (coated) | (MA133620) |
| GPR56 | 4C3 | Blocking | Biolegend (391902) |
| CD29 | P5D2 | Blocking | Biolegend (921304) |
| CD29 | TS2/16 | Agonist/blocking at | Invitrogen (14-0299- |
| | | high dose | 82) |
| CD49b | HAS-4 | Block | Southern Biotech |
| | | | (9426-01) |
| CD49b | AK7 | Agonist/blocking at | Invitrogen (MA5- |
| | | high dose | 43998) |
| CD49a | 5E8D9 | Blocking | Sigma (05-246) |
| Integrin αV | NKI-M9 | Blocking | Biolegend (327902) |
| (CD51) | | | |
| Integrin αV | 13C2 | Blocking (soluble)/ | Southern Biotech |
| (CD51) | | agonist (coated) | (9656-01) |
| Integrin β3 | VI-PL2 | Blocking | Biolegend (336402) |
| (CD61) | | | |

Other antibodies that block ECM components can also be identified, e.g., using binding assays and functional assay of NK cell activation and target killing in the presence of ECM proteins +/−antibody to determine the efficacy of antibody blockade.

### Inhibitors of Collagen Deposition or Production

The methods described herein can also include administering an inhibitor of collagen deposition or production, including losartan (e.g., losartan potassium) or 3,4-dihydroxybenzoic acid (DHB), LOX inhibitors (e.g., 2-aminopropionitrile), LOXL2 inhibitors (e.g., ellagic acid, PXS-5153A), antifibrotic drugs (e.g., Pirfenidone or Nintedanib, or Ormeloxifene); or TGFb inhibitors (e.g., SB431542).

### Dosage

An “effective amount” is an amount sufficient to effect beneficial or desired results. For example, a therapeutic amount is one that achieves the desired therapeutic effect, e.g., boosting NK cell cytotoxicity to increase anti-tumor and anti-viral immunity in a subject. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Pharmaceutical Compositions and Methods of Administration

The methods described herein include the use of pharmaceutical compositions comprising or consisting of (1) blocking antibodies to ECM components and/or (2) inhibitors of collagen deposition or production, as active ingredients. In some embodiments, the compositions include a combination of lair1 plus CD29 (CD49b and CD49a) antibodies.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### Examples

The invention is further described in the following examples, which do not limit the scope of the invention described in the claims.

### Materials and Methods

The following materials and methods were used in the Examples set forth below.

### Mice

C57BL/6 albino WT, B2m, m157^{tg}, m157^{tg}, B2m^{-/-}, C57BL/6 Ncr1^{iCre}, ROSA^{mT-mG}, Ncr1^{iCre}, Tgfbr2^{fl/fl}, Act-mOVA^{tg}, C57BL/6×BALB/c F1 Ncr1^{iCre}, ROSA^{mT-mG}, C57BL/6 Ncr1^{iCre}, ROSA^{DTR}, C57BL/6×BALB/c F1 Ncr1^{iCre}, ROSA^{DTR} and C57BL/6×BALB/c F1 CD4^{Cre}, ROSA^{DTR} were bred in house. C57BL/6 mice (Charles River, Wilmington, MA, USA, strain code: 207) were used in the parabiosis, LEGENDplex and tumor experiments. C57BL/6-Ly5.1 mice (Charles River) were used for in vivo BM rejection experiments. C57BL/6 Hobit^{-/-} mice and Lair1^{-/-} mice were kind gifts from KP van Gisbergen, Sanquin Research and Landsteiner Laboratory, Amsterdam UMC, Amsterdam, The Netherlands, and Svetlana Komarova, Faculty of Dentistry, McGill University, Quebec, Canada, respectively. Mutant mice were genotyped using the primers listed in table 1. All mice were housed under specific pathogen-free conditions, given water and food ad libitum, in the animal facility at Massachusetts General Hospital in accordance with animal care regulations. All mice were closely monitored by the authors, facility technicians and an independent veterinarian when necessary. All procedures were performed according to the protocols approved by the Institutional Animal Care and Use Committee (IACUC) at Massachusetts General Hospital.

### Skin Transplantation

C57BL/6 albino ear skin from WT, B2m^{-/-}, m157^{tg}, m157^{tg},B2m^{-/-} and Act-mOVA^{tg} mice was harvested after euthanasia, cartilage was removed and skin placed in a petri dish with 1×PBS (Life Technologies, Thermo Scientific, Grand Island, NY, USA, catalog no. 14190144). Recipient mice were anesthetized for the procedure with an intraperitoneal (i.p.) injection of 4.5 μL/g body weight of 10 mg/mL ketamine (Keta Ved®, Boehringer, Ingelheim Vetmedica, Fort Dodge, IA, USA, catalog no. 045-290) combined with 0.5 mg/mL xylazine (AnaSed®, Akorn, Lake Fortes, IL, USA, catalog no. 139-236) sterile solution in addition to isofluorane (Baxter Healthcare Corporation, Deerfield, IL, USA, catalog no. 1001936040) as required. Mice were placed on a heating pad during and after the surgery. Backs of recipient mice were shaved and cleansed with 70% ethanol. A circular piece of recipient skin approximately 1.5 times the area of ear skin to be transplanted was excised from the back of recipient mice and subcutaneous fascia was removed. Donor ear skin was placed split side down and sutured using n° 6-0 silk surgical sutures (Ethicon, Puerto Rico, catalog no. K889H). Approximately one dozen equidistant sutures were made around the donor skin to attach the ear to the recipient tissue. Next, the skin transplant was coated with antibiotic ointment (Medline Industries, Northfield, IL, USA, catalog no. CUR001231) and protected with a 2 cm×2 cm patch of Vaseline gauze (Convidien, Mansfield, MA, USA, catalog no. 884413605). A bandage (Tensoplast®, BSN medical, Charlotte, NC, USA, catalog no. 02115-00) was wrapped around the torso of the mouse and sutured in place using n° 4-0 silk surgical sutures (Ethicon, Puerto Rico, catalog no. K871) on the dorsal region suturing into the back skin adjacent to the shoulders and hips. Seven days post-surgery the bandage was removed, and donor skin was monitored daily for any sign of inflammation and/or rejection. Pictures were taken every other day and ImageJ (Version 1.52a) was used to measure graft size. Recipient and donor mice were sex and aged-matched.

### Tissue Harvesting

Mice were anesthetized using ketamine/xylazine and 1 μg of monoclonal anti-CD45-BV605 (clone 30-F11, BioLegend, San Diego, CA, catalog no. 103155) was injected by retroorbital injection to label circulating CD45⁺ cells. After three minutes had elapsed, peripheral blood was collected by retroorbital bleeding and, following euthanasia, liver, spleen, lymph nodes, donor and recipient skin were collected for analysis. Red blood cells in peripheral blood, spleen and liver were lysed using red blood cell lysis buffer (RBC lysis buffer 10X, Biolegend, catalog no. 420301). After washing with 1×PBS/2% FCS/5 mM EDTA, 5×10⁶ cells were prepared for flow cytometry staining.

### Skin Harvesting

The back of the mouse was shaved and a large rectangle of shaved skin encompassing the donor graft and recipient skin was excised. Large regions of fat on the underside of the skin were carefully scraped away then the donor and recipient skin were separated with a razor blade. These were placed in separate dishes and chopped with scissors into ˜1 mm pieces. Each sample was then placed into a 15 mL tube with 10 mL digestion buffer (RPMI 1640 (Life Technologies, catalog no. 21870076), 200 U/mL Collagenase IV (Worthington Biochemical, Lakewood, NJ, USA)). Skin was incubated for 2 hours at 37° C. with shaking. Following incubation, digested skin was poured onto a 70 μM strainer placed in a 50 mL tube. The 15 mL tube was rinsed with ˜3 mL of 1×PBS/2% FCS/5 mM EDTA and added to the digested sample. Skin was mashed with a plunger through the 70 μM strainer which was then rinsed ˜3 times with a small volume of 1×PBS/2% FCS/5 mM EDTA. The cells were centrifuged at 300 g for 5 minutes at 4° C. and resuspended in 2.4G2 blocking solution before flow cytometry staining. NK cells were stained for activating, inhibitory receptors and CD107a surface expression for baseline evaluation. For the ex vivo NK cell stimulation assay, donor skin graft derived cells were resuspended in RPMI/10% FBS/1% penicillin and streptomycin (P/S, Thermo Fisher, Waltham, MA, catalog no. 14140122)/20 mM Glutamine and were plated on control IgG (clone MOPC-173, Biolegend, catalog no. 400203) or aNK1.1 (clone PK136, Biolegend, catalog no. 108703) pre-coated plate (Nunc™ MaxiSorp™ plate, Biolegend, catalog no. 423501) or treated with 1×PMA/Ionomycin solution (Biolegend, catalog no. 423301). The cells were incubated at 37° C./5% CO₂ for total 4 hours with last 3 hours in the presence of 1 μL of anti-CD107a-BV421 (1D4B, Biolegend, catalog no. 121618), 1×Brefeldin A (Biolegend, catalog no. 420601) and 1×Monensin (Biolegend, catalog no. 420701). Following the co-culture, cells were washed with 1×PBS/2% FCS/5 mM EDTA and the pellet was resuspended in 2.4G2 blocking solution before flow cytometry staining.

### Liver Harvesting

The peritoneal cavity was opened to expose the liver and 5-10 mL of 1×PBS was injected into the hepatic portal vein using a 27G needle to perfuse the liver. The gall bladder was then removed and all lobes of the liver collected and mashed through a 70 μm filter sitting in a 50 mL tube using a syringe plunger. The filter was rinsed with 1×PBS/2% FCS/5 mM EDTA 3-4 times during the mashing procedure. The cell suspension was centrifuged at 300 g for 5 minutes at 4° C. then supernatant aspirated and the cell pellet washed with 10 mL of 1×PBS/2% FCS/5 mM EDTA. Following centrifugation and aspiration of supernatant the pellet was re-suspended in 25 mL of isotonic Percoll (8.44 mL Percoll [Healthcare Biosciences, Uppsala, Sweden, catalog no. 17-0891-01]/0.47 mL 20×PBS/16.09 mL 1×PBS) at room temperature (RT). Liver cell suspensions were centrifuged at 693 g for 12 minutes at RT with no brake, the leukocyte pellet at the bottom of the tube was then washed with 10 mL of 1×PBS/2% FCS/5 mM EDTA and red blood cells lysed (RBC lysis buffer 10X, BioLegend, catalog no. 420301). Following washing with 1×PBS/2% FCS/5 mM EDTA the pellet was resuspended in 2.4G2 blocking solution before flow cytometry staining.

### Flow Cytometry

Single-cell suspensions from all samples were prepared by straining through a 70 μm filter. 2.4G2 supernatant treated cells were stained in 1×PBS/2% FCS/5 mM EDTA with the appropriate surface antibodies (Table 2) for 30 minutes at 4° C., washed and analyzed by flow cytometry. For intracellular staining, cells were fixed and permeabilized using True-Nuclear Transcription Buffer set (Biolegend, catalog no. 424401) according to the manufacturer's protocol then incubated with appropriate antibodies (Table 2) for 60 min at RT, washed and analyzed by flow cytometry. For in vitro signaling experiments, following the specified time points, cells were fixed with an equal volume of pre-warmed 1×aldehyde based fixation buffer (Biolegend, catalog no. 420801) for 20 min at RT. Fixed cells were permeabilized by drop-wise addition of chilled methanol-based solution True-Phos Perm Buffer (Biolegend, catalog no. 425401) while mixed on a vortex. Cells were stored in-20° C. and dark for 1-3 days before washing them two times with ˜3 mL of 1×PBS/2% FCS/5 mM EDTA. Cells were subsequently stained with appropriate antibodies against surface markers and specific phosphorylated signaling proteins (Table 2) for 60 min at RT, washed and analyzed by flow cytometry. Cells were assayed on a BD LSRFortessa X-20 flow cytometer (BD Bioscience, Billerica, MA, USA) and data were analyzed using FlowJo software Version 10 (Tree Star, Ashlad, OR, USA). NK cells in WT non-reporting mice were identified as CD3⁻ NK1.1⁺NKp46⁺.

### Bone Marrow Transplantation

Bone marrow cells from B6-Ly5.1 (CD45.1), B6 WT (CD45.2) and B6 B2m^{-/-} (CD45.2) were collected from the tibia and femur by flushing bones with RPMI 1640 into a petri dish under sterile conditions. Cells were then counted and resuspended in RPMI 1640. Ly5.1:WT (control) and Ly5.1:B2m^{-/-} (test) BM cells were mixed 1:1 and intravenously injected into sub-lethally irradiated (450 cGy) Ncr1^{icre}, Rosa^{mT-mG} recipients. Three days later, spleens of recipient mice were harvested and the ratio WT:Ly5.1 and WT:B2m^{-/-} cells was determined by flow cytometry. % rejection was calculated as follows:

$$1 \; 00 - ( [ \frac{B 2 {m}^{- / -} / B 2 {m}^{+ / +} output}{B 2 {m}^{- / -} / B 2 {m}^{+ / +} input} ] \times 100 )$$

### Adoptive NK Cell Transfer

m157^{tg}, B2m/donor skin was transplanted onto the back of C57BL/6 WT mice following the above-mentioned skin transplant procedure. On day one post-transplant, six spleens from Ncr1^{iCre} ROSA^{mTmG} were harvested and mashed through a 70 μm filter. Spleens were resuspended in RBC lysis for two minutes and washed with 1×PBS/2% FCS/5 mM EDTA. NKp46-GFP⁻ ROSA cells were sorted using a Sony FX500 cell sorter (Sony Biotechnology, San Jose, CA, USA). Sorted cells were centrifuged and 8.5×10⁵ NKp46-GFP⁺ cells resuspended in 200 μL of sterile RPMI 1640 were filtered through a 40 μm strainer followed by intravenous injection into recipient mice using a sterile 28G syringe (0.5 mL BD insulin syringe, Franklin Lakes, NJ, USA, catalog no. 329461). Negative controls consisted of mice injected with 200 μL RPMI 1640 alone. 20 days post-transplant, mice were euthanized and peripheral blood, spleen, liver, donor and recipient skin were collected for analysis.

### Parabiosis

Parabiont partners were co-housed in the same cage for a week before the surgery. Eight to twelve week old female C57BL/6 WT mice were surgically connected to Ncr1^{iCre} ROSA^{mTmG} weight and age-matched partner female mice. Surgery was performed on a heating pad and animals anesthetized using isoflurane. Longitudinal skin incisions were made on the shaved sides of each animal. Knee and elbow joints from each animal were first sutured using n° 4-0 surgical suture. Following attachment of joints, the skin of the animals was connected using n° 6-0 sutures ending with a double surgical knot. To minimize pain, 0.1 mg of Carpofren (Rimadyl, Zoetis, Brazil, catalog no. 141-199) was injected I.P. each day for two days post-surgery, in addition to close monitoring every day for signs of pain or stress. On day 21 post-parabiosis, ear skin from m157^{tg} B2m^{-/-} was transplanted onto the back of the C57BL/6 WT mouse of each pair. After 20 days, mice were euthanized and peripheral blood, spleen, liver, donor and recipient skin from the C57BL/6 WT parabiont were collected for analysis.

### NK Cell Sorting

At day 20 post m157^{tg} B2m^{-/-} transplantation onto Ncr1^{iCre} ROSA^{mTmG} B6 mice and day 10 post m1574 g transplantation onto Ncr1^{iCre} ROSA^{mTmG} F1 mice, NK cells from blood, spleen and donor skin (cNK and trNK) and recipient skin (trNK cells) were sorted for RNA-sequencing. First, mice were anesthetized using ketamine/xylazine and 1 μg of monoclonal anti-CD45-BV605 (clone 30-F11, BioLegend) was injected via the retroorbital route three minutes before tissue harvest to label circulating CD45⁺ cells. Tissues were harvested as described above and, following staining, NKp46⁺ROSA⁻CD45⁺CD49a⁻CD49b⁺ (cNK) from peripheral blood and spleen, CD3⁻NKp46⁺ROSA⁻CD45⁻CD49a⁻CD49b⁺ (cNK), CD3⁻ NKp46⁺ROSA⁻CD45⁻CD49a⁺CD49b⁺ (dpNK), CD3⁻NKp46⁺ROSA⁻CD45⁻CD49a⁺CD49b⁻ (trNK) from donor skin and CD3⁻NKp46⁺ROSA⁻CD45⁻CD49a⁺CD49b⁻ (trNK) cells from recipient skin were sorted using a BD FACSAria II (BD Bioscience, Billerica, MA, USA). Sorted NK cells were collected in 15 mL tubes containing 3 mL of RPMI 1640 medium supplemented with 10% Fetal Bovine Serum (FBS, Corning, Manassas, VA, USA) and 5% P/S.

### RNA Sequencing and Analysis

Sorted cells were centrifuged, media aspirated and resuspended in 5 μL of TCL buffer with 1% β-mercaptoethanol (BME, Fisher Scientific, catalog no. 21-985-023) and added to a 96-well Eppendorf twin-tec barcoded plate (Eppendorf, NY, USA). Plates were stored at −80° C. until sequencing as described (60). Modified SmartSeq2 cDNA and Illumina Nextera XT library construction and sequencing were conducted at the Broad Institute of MIT and Harvard using an Illumina NextSeq 500 System (Boston, MA, USA). All samples were quasi-mapped to GRCm38 (mm10) using Salmon with the “gcBias” and “seqBias” options and collapsed down to gene-level abundance estimates using the “EnsDb.Mmusculus.v79” annotation package. All downstream differential expression analysis was carried out using DESeq2. The results table was restricted to genes with a minimum of 10 total counts across the dataset, and mitochondrial, pseudo-, and ribosomal genes were removed. Original data are available at the NCBI Gene Expression Omnibus (GEO), accession numbers: GSE148600. Gene set enrichment analysis (GSEA) (broad.mit.edu/gsea/) was used to identify significantly up- or down-regulated pathways in CD49b⁺ cNK cells in donor skin in comparison with CD49⁺ cNK cells in circulation (blood and spleen). GSEA analysis was performed using MSigDB 50 hallmark gene sets (h.all) and the curated 6226 gene sets (c2.all) and pathways containing enriched genes between 15 and 1000 genes were considered.

### Cell Lines

B16-F10 wildtype (WT) and B2m^{-/-} cell lines were maintained in RPMI 1640 media supplemented with 10% FBS and 1% P/S and cultured at 37° C./5% CO₂. WT Mouse Embryonic Fibroblasts (WT-MEF) and m157-expressing MEF (m157-MEF) were a gift from Wayne Yokoyama, Washington University, St Louis, MO, USA. MEFs were cultured in RPMI 1640 media, containing 10% FBS, 1% P/S/20 mM Glutamine (Life Technologies, catalog no 25030-081).

### Melanoma Experiments

All tumor experiments were performed in eight to twelve-week-old C57BL/6 female WT or Ncr1^{iCre} ROSA^{mT-mG} mice. For the S.C. tumor model, 2.5×10⁵ B16-F10 WT or B2m^{-/-} cells were subcutaneously injected into the shaved flanks of mice. Tumor growth was measured every other day using a digital caliper starting from day seven. Tumor volume was calculated as follows:

$$volume ( {cm}^{3} ) = \frac{( lenght \times {width}^{2} )}{2} .$$

To inhibit collagen deposition in and around the tumor, mice were administered with 60 mg/kg of Losartan Potassium (Fisher Scientific, catalog no. L02325G) or 40 mg/kg of 3,4-dihydroxybenzoic acid (DHB) (Sigma-Aldrich, catalog no. 37580) in 100 μL of sterile 1×PBS while the control mice were administered with the equivalent volume of sterile 1×PBS. These drugs were injected I.P. every day for the duration of the study starting the day of tumor inoculation.

For the collagenase/hyaluronidase experiment, 2.5×10⁵ B16-F10 B2m^{-/-} cells were resuspended in 1×collagenase and hyaluronidase mix (Stemcell, catalog no. 07912) and were subcutaneously injected into the shaved flanks of mice. To deplete collagen within the tumor, mice were administered with 1×collagenase and hyaluronidase mix (Stemcell, catalog no. 07912) in 100 μL of sterile 1×PBS while control mice were administered with the equivalent volume of sterile 1×sPBS. These enzymes were injected via intratumoral (I.T.) route starting on the day of tumor inoculation (day 0), day 1, day 2, day 3, day 4 and then every other day (day 6, 8, 10, etc.) until the end of the study. Except for day 3 tumor harvest, mice were euthanized when a tumor reached 2 cm in diameter in accordance with IACUC protocols. For early time point tumor analysis, specific Ncr1^{iCre}, ROSA^{mT-mG} mice from PBS, Losartan and DHB group were euthanized on day 3 post tumor inoculation, the implanted tumor harvested and fixed in pre-chilled 95% ethanol overnight at 4° C. before subjecting them to subsequent processing.

For metastatic melanoma studies, mice were intravenously injected via tail vein with 2×10⁵ B16-F10 WT or B2m-cells. Mice were euthanized 14 days post-injection, lungs were then harvested, fixed in 4% Paraformaldehyde (PFA, Sigma Aldrich, catalog no. P6148) and collected for histological analysis. Blinded quantification of the metastatic lung foci was performed on macroscopic images and the size of metastatic foci was measured using ImageJ software.

### Tumor Harvesting

Terminal subcutaneous tumors were excised with the skin and weighed before cutting in half. One half was placed in appropriate fixative while the other half was separated from the skin and chopped with scissors into ˜1 mm pieces. These were transferred to a 15 mL tube with 10 mL digest buffer (RPMI 1640 (Life Technologies, catalog no. 21870076), 200 U/mL Collagenase IV (Worthington Biochemical, Lakewood, NJ, USA)) and incubated for 2 hours at 37° C. with shaking. Following incubation, digested tumor was poured onto a 70 μM strainer placed in a 50 mL tube. The 15 mL tube was rinsed with ˜3 mL of 1×PBS/2% FCS/5 mM EDTA and added to the digested sample. The tumor was mashed with a plunger through the 70 μM strainer which was then rinsed ˜3 times with a small volume of 1×PBS/2% FCS/5 mM EDTA. The tumor-infiltrating CD45⁺ immune cells were isolated by magnetic-based positive selection using CD45 microbeads (Miltenyi Biotec, catalog no. 130-052-301) according to the manufacturer's protocol. The stained cells were passed through the LS columns (Miltenyi Biotec, catalog no. 130-042-401) placed between magnets for isolation of immune cells. The isolated CD45⁺ cells were centrifuged at 300 g for 5 minutes at 4° C. and resuspended in 2.4G2 blocking solution before flow cytometry staining.

### Cytokine, Poly(I:C) and DT Treatment

For cytokine treatment, mice were injected with 100 ng/mouse of mIL-12 (Biolegend, catalog no. 577002), 2 μg/mouse of mIL-15 (Shenandoah Biotechnology, Warwick, PA, catalog no. 200-07) and 100 ng/mouse of mIL-18 (Shenandoah Biotechnology, catalog no. 200-83) in 200 μL of sterile 1×PBS. For the combination Poly(I:C) and cytokines, mice were injected with 200 μg/mouse of Poly(I:C) (Sigma-Aldrich, catalog no. P9582), 2 μg/mouse of mIL-15 (Shenandoah Biotechnology, catalog no. 200-07) and 100 ng/mouse of mIL-18 (Shenandoah Biotechnology, catalog no. 200-83) in 200 μL of sterile 1×PBS. Treatments were injected I.P. starting the day of the skin transplant (day 0), day 2 and day 4 and then injected S.C. at days 7, 10, 13, 16 and 19 post-transplant. Diphtheria toxin (DT, Sigma-Aldrich, catalog no. D0564) was injected I.P. at 500 ng/mouse the day before the transplant and 200 ng/mouse at days indicated in FIG. S5E combined with NK or T cell depletion.

### NK Cell Depletion, T Cell Depletion and Checkpoint Blockade

Mice were I.P. injected with 500 μg/mouse of IgG isotype control (Southern Biotech, Birmingham, AL, USA, catalog no. 0107-01) or depleting anti-NK1.1 antibody (clone PK136, BioXcell, West Lebanon, NH, USA, catalog no. BE0036) two days before tumor cell injection and 250 μg/mouse every other day starting the day of the tumor cell injection. For depletion of NK or T cells (anti-CD4 antibody, clone GK1.5, BioXcell; anti-CD8a antibody, clone YTS 169.4, BioXcell) in a skin transplant setting, 500 μg/mouse of each depleting antibody were injected the day of the transplant and 250 μg at days indicated in FIG. S5E. Anti-mouse PD-1 (clone 29F.1A12, BioXCell, catalog no. BE2073), anti-TIGIT (clone 1G9, BioXCell, catalog no. BE0274) and anti-CTLA4 (clone 9D9, BioXCell, catalog no. BE0164) antibodies were I.P. injected at 200 μg each/mouse at day-2 then every three days beginning at day 0 of skin transplantation.

### In Vitro Experiments

Prior to the experiment, blood from C57BL/6 WT and m157^{tg} mice was collected, incubated overnight at 4° C., centrifuged and serum was isolated from the pellet. Spleens from C57BL/6 mice were harvested, mashed through a 70 μm filter and cells were resuspended in RBC lysis for two minutes. After washing with 1×PBS/2% FCS/5 mM EDTA, cells were counted and 5×10⁶ splenocytes resuspended in 500 μL RPMI 10% FBS/1% P/S/20 mM Glutamine and 10% serum of either WT or m157^{tg} mice then plated in a 24-well plate and incubated for 3 h at 37° C./5% CO₂. Cells were then washed, re-plated and 200 μL of a mixture containing 1×10⁵ WT-MEF or m157-MEF, mIL-12 (10 ng/mL, Biolegend, catalog no. 577002) or mIL-12/mIL-18 (12.5 ng/mL, Shenandoah Biotechnology, catalog no. 200-83) was added to the well. After incubation for another hour at 37° C./5% CO₂, 1 μL of anti-CD107a-eF660 (clone 1D4B, eBioscience, catalog no. 50-1071-82), 1×Brefeldin A (Biolegend, catalog no. 420601) and 1×Monensin (Biolegend, catalog no. 420701) were added and cells were incubated for 6 hours. Then, the supernatant was removed, cells were washed and resuspended in 2.4G2 blocking solution before flow cytometry staining. Cells were assayed on a BD FACSCanto (BD Biosciences).

For in vitro experiments in the presence of ECM components, Collagen I (Rat tail, Sigma, catalog no. 08-115), collagen III (Human placenta, Sigma, catalog no. C4407), collagen IV (Mouse, Sigma, catalog no. C0543), Elastin (Mouse lung, Sigma, catalog no. E6402), Laminin (Mouse, Thermo Fisher, catalog no. 23017015) and Fibronectin (Rat plasma, Sigma, catalog no. F0635) were diluted in 1×PBS and coated at 20 μ ENREF 45 g/cm² in a 24 well tissue culture plate for 30 minutes at RT with gentle shaking followed by overnight at 4° C. Spleens from Ncr1^{iCre}, ROSA^{mT-mG}, WT or Lair1^{-/-} mice were harvested as described above. Then, 5×10⁶ splenocytes resuspended in RPMI 10% FBS/1% P/S/20 mM Glutamine were mixed with 20 μg of respective ECM protein per well and were incubated at RT for 10 minutes. Splenocytes and ECM mix were plated onto the ECM-coated well and were incubated at 37° C./5% CO₂ for 2 hours (short ECM exposure) or 17 hours (long ECM exposure). At the end of the incubation, 1×10⁵ MEF (WT or m157) cells were added to each well to obtain a 50:1 splenocyte:MEF ratio. For cell signaling experiments, hydrogen peroxide (H₂O₂) at a final concentration of 2.5 mM or 1×10⁵ MEF-m157 cells were added to each well except in “0 min” wells which were unstimulated and fixed immediately. Splenocytes were stimulated with 2.5 mM H₂O₂ for 2 min and 5 min or were co-cultured with MEF-m157 for 30 min and 60 min (signaling) or 7 hours (CD107a and IFN□) at 37° C./5% CO₂. Splenocytes and MEF in co-culture were supplemented with mIL-12 (10 ng/mL, Biolegend, catalog no. 577002), mIL-15 (1 ng/mL, Shenandoah Biotechnology, catalog no. 200-07) and hIL-2 (Biolegend, catalog no. 589104; only long ECM exposure) as well as for CD107a/IFN experiments. 1 μL of anti-CD107a-BV421 (1D4B, Biolegend, catalog no. 121618), 1×Brefeldin A (Biolegend, catalog no. 420601) and 1×Monensin (Biolegend, catalog no. 420701) were added. Following the co-culture, cells were washed with 1×PBS/2% FCS/5 mM EDTA and the pellet was resuspended in 2.4G2 blocking solution before flow cytometry staining.

### NK Cell Enrichment

NK cells purified from spleens under sterile conditions were used for LEGENDplex experiments. Spleens from 8-9-week-old C57BL/6 female WT mice were harvested and mashed through a 70 μm filter. Cells were resuspended in RBC lysis for two minutes and washed with 1×PBS/2% FCS/5 mM EDTA. NK cells were enriched by negative selection using an NK cell isolation kit (Milteny Biotec, catalog no. 130-115-818) and magnetic LS columns (Milteny Biotec, catalog no. 130-042-401) according to the manufacturer's instructions. Enrichment of isolated NK cells was confirmed by flow cytometry and was 88-90% CD3⁻NK1.1⁺NKp46⁺ cells. Enriched NK cells were resuspended in RPMI 1640 media supplemented with 10% FBS and 1% P/S.

### LEGENDplex

ECM protein coating on wells was performed as previously described. 1×10⁵ enriched splenic NK cells was mixed with 20 μg of respective ECM protein per well in RPMI 1640/10% FBS/1% P/S/20 mM Glutamine and incubated at RT for 10 minutes. NK cells and ECM mix were plated onto the ECM-coated well and were incubated at 37° C./5% CO₂ for 2 hours. At the end of the incubation, 1×10⁵ MEF (WT or m157) cells were added to the respective wells to obtain a 1:1 NK:MEF ratio. NK cells and MEF were co-cultured at 37° C. 5% CO₂ for 24 hours supplemented with mIL-12 (10 ng/mL, Biolegend, catalog no. 577002) and mIL-15 (1 ng/ml, Shenandoah Biotechnology, catalog no. 200-07-100 μg). The supernatant was collected at 12- and 24-hour intervals, centrifuged to remove cells/cell debris and subsequently frozen at −80° C. until further use. Quantification of murine cytokines and chemokines (CCL2, CCL5, CXCL9, CXCL10) in supernatants was performed with LEGENDplex kits (Biolegend, catalog nos. 740451, 740622) according to manufacturer's instructions. In brief, a mix of capture beads with distinct size and fluorescence was incubated with undiluted S/N or standard dilutions for 2 hours at RT with constant shaking. Beads were washed, a biotinylated detection antibody was added and the plate was incubated for 1 hour at RT with constant shaking. The streptavidin-PE conjugate was added and further incubated for 30 minutes at RT on a shaker. Beads were washed and subsequently acquired on a BD LSRFortessa X-20 flow cytometer. Analysis and quantification of the results were done using LEGENDplex data analysis software (BioLegend). Quantification was reported as pg/ml for CCL2, CCL5, CXCL10 and as MFI for CXCL9.

### Histology, Collagen, Elastin and Immunofluorescence Staining

Skin grafts were collected and incubated in 4% PFA/25% sucrose (Fisher Scientific, Hampton, NH, USA, catalog no. S5-3) solution at 4° C. for 16 hours. The next day, slices were equilibrated for 4 h in 50% sucrose solution, embedded in OCT (Fisher Scientific, catalog no. 23-730-571), snap frozen and stored at −80° C. For paraffin embedding, lungs and tumor were collected and fixed in 4% PFA overnight at 4° C. Subsequently, tissues were dehydrated in ethanol, processed and embedded in paraffin according to standard histology processes. For paraffin embedding of Ncr1^{icre} Rosa^{mT-mG} a different fixation protocol was used to maintain the NKp46-GFP and Tdt-Tomato-ROSA fluorescence. Tumors from Ncr1^{icre}, Rosa^{mT-mG} mice were collected and fixed in pre-chilled 95% ethanol overnight at 4° C. Tissues were subsequently dehydrated in 100% ethanol, cleared in 100% xylene and embedded in paraffin according to standard histology processes. For IF staining of frozen tissue, 7 μm sections of skin were cut on a cryostat, re-hydrated in three washes of 1×Tris-buffered-saline (TBS) for 2 minutes each followed by blocking of nonspecific protein with 5% bovine serum albumin (BSA, Fisher Scientific, catalog no. BP1600) and 5% goat serum (Sigma-Aldrich, catalog no. G9023) in 1×TBS. Sections were stained overnight at 4° C. with primary antibodies (Table 2). The following day, slides were washed as above and incubated for two hours at RT with secondary antibodies conjugated to fluorochromes (Table 2). After washing as above, sections were incubated with a 1:4000 dilution of DAPI (Invitrogen, Carlsbad, CA, catalog no. D3571) in 1×TBS for 5 minutes at RT, washed, air-dried and coverslips mounted with Prolong Gold Antifade Reagent (Invitrogen, catalog no. P36930). Five or six randomly selected fields of view at 200× total magnification were obtained for each section using a Zeiss Axio Scan (Zeiss, Oberkochen, Germany). Blinded manual counting of NKp46⁺, CD3⁺, CD4⁺ and CD8⁺ T cells were performed using ZEN Blue Software (Zeiss, Oberkochen, Germany). Automated counting was performed for CD11b, F4/80 and Arginasel stains in the whole tumor region of the scanned slides by Halo3.0 software (Indica Labs, Albuquerque, USA). For IF of paraffin-embedded tissues, 5 μm sections were rehydrated and permeabilized with 1×PBS supplemented with 0.2% Triton X-100 (Thermo Fisher Scientific, catalog no. BP151) for 5 minutes. Antigen retrieval was then performed using a Cuisinart pressure cooker for 20 minutes at high pressure in antigen unmasking solution (Vector Laboratories, Burligame, CA, catalog no. H-3300). Slides were then washed three times for three minutes each in 1×PBS supplemented with 0.1% Tween 20 (Sigma-Aldrich, catalog no. P1379). Sections were blocked, stained (Table 2) and mounted as described above. Elastin fiber staining was performed using Verhoeff Van Gienson Elastin Stain kit (ab 150667, Abcam) following the manufacturer's protocol. For collagen staining, Masson's Trichrome staining (Polysciences, Warrington, PA, catalog no. 25088) was performed following the instructions of the manufacturer. Whole slide imaging was performed using a NanoZoomer S60 Digital slide scanner (Hamamatsu, Japan) or Axio Scan.Z1 (Zeiss, Germany) and analyzed with NDP-view2 software (Hamamatsu) or ZEN Blue Software (Zeiss, Oberkochen, Germany), respectively.

### B2M Analysis in Human Cancers

B2M gene alterations and expression in solid cancer and leukemia samples in TCGA and other public databases were obtained and analyzed through cBioPortal for Cancer Genomics at cbioportal.org.

### Statistical Analysis

Graphs show mean values±standard deviation (SD). The numbers of mice per group used in each experiment are annotated in the corresponding figure legend as n. Graphs and statistical analysis were performed using GraphPad Prism 8 (La Jolla, CA, USA) and RStudio. All tumor quantifications were performed blindly. Two-tailed Fisher's exact test was used to compare skin graft rejection grades and lymph node metastatic load among groups. Two-way ANOVA with Sidak's multiple comparison test was used to compare tumor growth over time between different groups. Comparisons of survival were performed with the Log-rank test. Two-tailed Mann-Whitney U test was used for all the other comparisons. A P value of less than 0.05 was considered significant.

TABLE 1
PCR Primers for Mouse Genotyping

| Gene | | |
|---|---|---|
| Name | Sequence | # |
| m157 | forward: TCAACTTCCGACGCAAAAGAAAT | 1 |
| | reverse: ACCGTCGATTCGTCAGTAACG | 2 |
| B2m | forward wild type: | 3 |
| | ACGGTGCAGACTGAGCTCTGTTTTCATCTGTCTTC | |
| | reverse: | 4 |
| | CAGGCGTATGTATCAGTCTCAGTGGGGGTGAATTC | |
| | forward mutant: | 5 |
| | TCAGGATGATCTGGACGAAGAGCATCAGGG | |
| | reverse: | 6 |
| | CAGGCGTATGTATCAGTCTCAGTGGGGGTGAATTC | |
| Act- | forward: CTTCCAGGATTCGGAGACAG | 7 |
| mOVA | reverse: TCTGAAAGGCATTGCTTGTG | 8 |
| Tgfbr2 | forward: TATGGACTG GCTGCT TTTGTA TTC | 9 |
| | reverse: TGGGGATAGAGGTAGAAAGACATA | 10 |
| Hobit | forward: TGCCTCCTTTTGACCCATGACATCCAAATT | 11 |
| | reverse: CTCTTTCTGGCTCATTTCCCATCTCCTCTC | 12 |
| | mutant: ATCAGTTCGCTTCTCGCTTC | 13 |
| Lair1 | forward wild type: GGTTTGAGGACAGACAGATGACC | 14 |
| | reverse: TTTATGAGGAGGACGACTGAGC | 15 |
| | forward mutant: CCAGGTGCACATGATCAGC | 16 |
| | reverse: TTTATGAGGAGGACGACTGAGC | 17 |

#, SEQ ID NO:

TABLE 2
Antibodies for Tissue Analysis

| Immunostaining | | |
|---|---|---|
| Antibodies | Clone | Source |
| Rat Anti-Mouse CD3 | CD3-12 | Abcam, Cambridge, MA |
| Rabbit Anti-Mouse CD8 | D4W2Z | Cell Signaling Technology, Danvers, |
| | | MA |
| Rabbit Anti-Mouse CD4 | EPR19514 | Abcam |
| Rabbit Anti-mouse vimentin | EPR3776 | Abcam |
| Rabbit Anti-Mouse CD11b | EPR19387 | Abcam |
| Rabbit Anti-Mouse F4/80 | D2S9R | Cell Signaling Technology |
| Rabbit Anti-Mouse | D4E3M | Cell Signaling Technology |
| Arginase1 | | |

Secondary Antibodies and staining kits

| | |
|---|---|
| Goat anti-Rat IgG, Alexa Fluor ® 647 | Thermo Fisher Scientific, Waltham, |
| conjugate | MA |
| Goat anti-Rabbit IgG, Alexa Fluor ® 568 | Thermo Fisher Scientific |
| conjugate | |
| Goat anti-Rabbit IgG, Alexa Fluor ® 488 | Thermo Fisher Scientific |
| conjugate | |
| Elastic stain Kit - Verhoeff Van Gienson | Abcam |
| (EVG) Stain | |
| Masson's Trichrome Stain Kit | Polysciences, Warrington, PA |

Flow Cytometry Antibodies
Mouse

| | | |
|---|---|---|
| CD3-eF450 | 145-2C11 | BioLegend |
| CD3-AF700 | 145-2C11 | BioLegend |
| CD3-PerCP-Cy5.5 | 145-2C11 | BioLegend |
| CD3-PE | 145-2C11 | BioLegend |
| CD3-PE-Cy7 | 145-2C11 | BioLegend |
| CD3-APC-eF780 | 145-2C11 | eBioscience |
| CD3-BUV395 | 17A2 | BD-Biosciences |
| CD4-eF450 | GK1.5 | BioLegend |
| CD4-PerCP-Cy5.5 | GK1.5 | BioLegend |
| CD4-AF700 | RM4-5 | BioLegend |
| CD8-AF700 | 53-6.7 | BioLegend |
| CD8-APC-Cy7 | 53-6.7 | BioLegend |
| CD8-BUV395 | 53-6.7 | BD-Biosciences |
| CD45-BV605 | 30-F11 | BioLegend |
| CD45-PerCP-Cy5.5 | 30-F11 | BioLegend |
| CD45.1-APC-eF780 | A20 | eBioscience |
| CD45.2-eF450 | 104 | eBioscience |
| CD19-PE-Cy7 | 1D3 | eBioscience |
| CD19-APC-Cy7 | 6D5 | BioLegend |
| CD49a-PerCP-Cy5.5 | Ha31/8 | BioLegend |
| CD49b-APC | DX5 | BioLegend |
| CD49b-APC-Cy7 | DX5 | BioLegend |
| NK1.1-PE | PK136 | BioLegend |
| NK1.1-PECy7 | PK136 | BioLegend |
| NK1.1-APC | PK136 | BioLegend |
| NK1.1-PerCP-Cy5.5 | PK136 | eBioscience |
| NKp46-FITC | 29A1.4 | BioLegend |
| NKp46-PE | 29A1.4 | BioLegend |
| NKp46-BV610 | 29A1.4 | BioLegend |
| NKG2D-APC | CX5 | eBioscience |
| NKG2A-APC | 16A11 | BioLegend |
| CD11b-BV395 | M1/70 | eBioscience |
| CD48-AF700 | HM48-1 | BioLegend |
| CD69-eF450 | H1.2F3 | BioLegend |
| CD69-AF647 | H1.2F3 | BioLegend |
| DNAM1-PE-Cy7 | 10E5 | BioLegend |
| FoxP3-AF700 | MF-14 | BioLegend |
| Ly49H-AF647 | 3D10 | BioLegend |
| Ly49H-PE | 3D10 | BioLegend |
| Ly49H-PE-Cy7 | 3D10 | BioLegend |
| Ly49C/I-BV605 | 5E6 | BD-Biosciences |
| Ly49A-Pacific Blue | YE1/48.10.6 | BioLegend |
| CD103-AF647 | 2.00E+07 | BioLegend |
| CD107-eF660 | 1D4B | eBioscience |
| CD107a-BV421 | 1D4B | BioLegend |
| TRAIL-PE | N2B2 | BioLegend |
| TGFBRII-APC | Polyclonal | R&D systems, Minneapolis, MN |
| TIGIT-AF700 | 2190A | R&D systems |
| IL4rα-PE-Cy7 | I015F8 | Biolegend |
| Ccr7-PE-Cy7 | 4B12 | Biolegend |
| IFNγ-eF450 | XMG1.2 | eBioscience |
| IFNγ-AF700 | XMG1.2 | BioLegend |
| IFNγ-PE-Cy7 | XMG1.2 | eBioscience |
| IFNγ-BV605 | XMG1.2 | BioLegend |
| TNFα-BV605 | MP6-XT22 | BioLegend |
| Granzyme B-AF647 | GB11 | BioLegend |
| Granzyme B-Pacific Blue | GB11 | BioLegend |
| CD279 (PD-1)-BV605 | 29A.1A12 | BioLegend |
| Phospho-PLCg1 (Tyr783)- | D6M9S | Cell Signaling Technology, |
| AF647 | | Danvers, MA |
| Phospho-PLCg1 (Tyr783)-PE | A17025A | BioLegend |
| Phospho-ERK1/2 | 6B86B9 | BioLegend |
| (Thr202/Tyr204)-BV421 | | |
| Phospho-NFkB p65 (Ser536)- | 93H1 | Cell Signaling Technology, |
| AF647 | | Danvers, MA |
| Phospho-STAT3 (Tyr705)- | 13A3-1 | BioLegend |
| BV421 | | |
| Phosho-STAT5 (Tyr695)- | SRBCZX | Thermo Fisher Scientific |
| PerCP-eFluor710 | | |

### Example 1. NK Cell-Activating Ligand Together with Loss of MHC-I is Insufficient to Induce Rejection of Syngeneic Skin Graft

In order to assess the requirements for NK cell cytotoxicity in solid organs, we developed a syngeneic skin transplant model. Ear skin from donor mice on an albino B6 background was transplanted onto the back of B6 recipients. WT and m157^{tg} mice with or without the deletion of beta-2 microglobulin (B2m) gene were used as donors. Ncr1^{iCre}, ROSA^{mT-mG} reporter mice expressing green fluorescent protein (GFP) in NKp46⁺ NK cells and TdTomato in all other cells were used as recipients (FIG. 1A) (26). This approach enabled us to examine the impact of an NK cell-specific activating ligand, loss of MHC-I (through B2m deletion) or the combination of both on skin transplant outcomes while precisely distinguishing donor from recipient skin by skin graft color (albino ear skin) and histologically by immunofluorescence (IF) imaging. WT, B2m^{-/-}, m157^{tg}, and m157^{tg}, B2m^{-/-} skin grafts were monitored for signs of inflammation and rejection over time. At 20 days post-transplant, B2m^{-/-} skin grafts showed no signs of rejection (FIGS. 1B and C). In contrast, B2m^{-/-} BM transplants were readily rejected in WT B6 recipients. m157^{tg} and m157^{tg}, B2m^{-/-} skin grafts displayed minor signs of inflammation, which was most apparent in the latter (FIGS. 1B and C), yet no significant change in the size of the skin grafts was observed between the four groups from day 10 to day 20 post-transplant (FIG. 1D). Monitoring m157^{tg}, B2m^{-/-} skin grafts out to day 60 post-transplant revealed no additional changes. Massive numbers of NKp46-GFP⁺ NK, CD4⁺ T and CD8⁺ T cells infiltrated the skin and the epidermis of m157^{tg} and m157^{tg}, B2m^{-/-} compared with WT grafts (FIGS. 1E and F). B2m skin grafts also showed higher NK and T cell infiltrates compared with WT grafts (FIGS. 1E and F). No NK or T cell infiltrate was detected in the recipient skin adjacent to the skin grafts across the four groups. These findings establish that the expression of NK cell-specific activating ligand, m157, in the skin grafts results in markedly enhanced recruitment of NK cells and an associated increase in T cell numbers in the grafts but does not lead to graft rejection.

Next, we examined the strength of m157-Ly49H interaction in NK cells that infiltrated the skin grafts. Recipient-derived CD49b⁺ cNK and CD49a⁺ trNK cells were found in m157-expressing skin grafts, which expressed Ly49H and exhibited m157-induced Ly49H downregulation compared with Ly49H⁻ and Ly49H⁻ cNK and Ly49H⁻ trNK cells in the recipient's liver (27). No significant alteration in NKG2D, CD48, DNAM1, NKG2A, Ly49A and Ly49C/I expression on the surface of CD49b⁺ cNK and CD49b″CD49a double-positive NK (dpNK) cells in m157^{tg}, B2m^{-/-} skin graft was detected. Although a large number of NKp46-GFP⁺ cNK and trNK cells infiltrated m157-expressing skin grafts, these cells did not upregulate IFNγ, TNFα or granzyme B expression. NK cells isolated from blood, spleen and lymph nodes of WT recipients transplanted with m157^{tg} skin did not downregulate Ly49H expression in contrast to those of m157^{tg} mice, ruling out systemic NK cell hyporesponsiveness in recipient animals (27). In addition, WT NK cells were fully responsive to m157 stimulation in the presence of serum from m157^{tg} mice in vitro, ruling out the possibility that a soluble form of m157 blocked NK cell activation in m157-expressing skin grafts. NK cells can become hyporesponsive upon chronic exposure to the loss of MHC-I (28, 29). To investigate this concept in our transplant system, we tested the ability of m157^{tg}, B2m^{-/-} skin graft-infiltrating NK cells at day 10 post transplantation to degranulate and produce IFNγ in response to ex vivo stimulation. Compared with splenic cNK cells, skin graft-derived cNK cells had comparable expression levels of MHC-I specific inhibitory receptors and were not found to be degranulating at baseline. However, upon ex vivo co-culture, Ly49H⁺ cNK cells from m157^{tg}, B2m^{-/-} skin graft showed robust degranulation and IFNγ secretion compared with splenic Ly49H⁻ cNK cells in control IgG-coated, aNK1.1 antibody-coated plates as well as upon phorbol myristate acetate plus ionomycin (PMA/Ion) stimulation. We did not observe any increase in degranulation and IFNγ secretion by Ly49H cNK cells in the skin graft versus spleen except for a minor increase in degranulation of skin Ly49H⁻ NK cells in IgG-coated plate. These findings demonstrate that Ly49H″ cNK cells from m157^{tg}, B2m^{-/-} skin graft are not hyporesponsive and are fully capable of activation and degranulation outside of the skin.

To investigate the timing of inflammation in m157-expressing skin grafts, we compared m157 g grafts to skin grafts expressing a model T cell antigen, Ovalbumin (OVA). WT B6 mice were transplanted with m157^{tg}, m157^{tg}, B2m^{-/-}, mOVA^{tg} and mOVA^{tg}, B2m^{-/-} skin grafts and monitored. As expected, mOVA^{tg} donor skin grafts were mostly rejected by day 20 post-transplant while the absence of MHC-I inhibited the rejection of mOVA^{tg}, B2m grafts (FIG. 1G). The paradoxical increase in inflammation in m157^{tg}, B2m^{-/-} compared with m157^{tg} grafts supported the dominant role of NK rather than T cells in initiating the immune response in m157-expressing grafts. However, we noticed that inflammation in m157^{tg},B2m^{-/-} skin grafts developed after day 12 post-transplant, along a similar adaptive immune response timeline to mOVA^{tg} grafts (FIG. 1G). These data suggest that early NK cell activation following m157% skin transplantation may initiate a delayed T cell response leading to skin inflammation. Nonetheless, NK cell activation against syngeneic skin expressing m157 and lacking MHC-I is insufficient to cause skin graft rejection.

### Example 2. cNK Cells Recruited from the Circulation into Skin Grafts Give Rise to trNK Cells and Fail to Reject the Graft Upon Additional Cytokine Stimulation

To overcome the apparent inhibition of cytotoxicity experienced by NK cells in m157-expressing skin grafts, we treated the recipient mice with IL-15, IL-18 and a TLR3 agonist, poly(I:C), or IL-12, IL-15 and IL-18 (FIG. 2A). These NK cell survival and activating factors are commonly used to enhance NK cell cytotoxicity (30, 31). However, m157^{tg} and m157^{tg}, B2m^{-/-} skin grafts treated with these agents showed no sign of rejection at day 21 post-transplant. Although the combination of IL-12, IL-15 and IL-18 had no effect on NK cell recruitment and their activation and maturation profile, these cytokines appeared to enhance the conversion of cNK cells into trNK cells in the skin graft, which may be mediated by IL-15 (FIGS. 2B-E) (32, 33). To elucidate the basis for this profound lack of NK cell cytotoxicity, we investigated the phenotype of CD49b⁺ cNK and CD49a⁺ trNK cells, which were highly enriched in m157^{tg}, B2m^{-/-} skin grafts. Recipient liver cNK cells were largely negative for CD69, CD103 and TRAIL, while the large majority of liver trNK cells expressed CD69 and TRAIL. cNK cells in m157^{tg}, B2m^{-/-} skin grafts expressed CD69 with minimal CD103 and TRAIL expression. In contrast, two-thirds of trNK cells in the skin graft co-expressed CD69 and CD103 supporting their tissue-resident status. Surprisingly, trNK cells in the skin graft expressed low levels of TRAIL. Combining the ratio of NK cell subtypes from flow cytometry data with absolute NK cell counts in histological sections showed that CD49b⁺ cNK cells were significantly increased in B2m^{-/-}, m157 g and m157^{tg}, B2m^{-/-} skin grafts compared with WT grafts (FIG. 2F). In addition, CD49a⁺ trNK cells and CD49b⁺CD49a⁻ dpNK cells were markedly increased in m157^{tg} and m157^{tg}, B2m^{-/-} skin grafts compared with WT grafts, which largely lacked trNK and dpNK cells (FIG. 2D). In contrast to cNK cells in WT skin grafts, all three NK cell subsets in m157^{tg}, B2m^{-/-} skin grafts expressed Ly49H at levels indicative of Ly49H downregulation upon binding to m157 on the donor skin cells (FIG. 2G). Labeling the circulating immune cells with intravenous (I.V.) injection of anti-CD45 antibody further demonstrated that the majority of cNK cells in WT and m157^{tg} skin grafts were CD45 negative and had exited circulation similar to trNK cells in the skin. Considering that trNK cells do not migrate (34-36) and ILCP-derived trNK cells are Ly49H negative (36), the large population of NKp46-GFP⁺Ly49H⁻ trNK cells in m157-expressing skin grafts indicates that circulating cNK cells give rise to trNK cells in the donor skin.

To determine whether NKp46-GFP⁺ NK cells in m157^{tg}, B2m^{-/-} skin grafts were recruited from circulation or the surrounding recipient skin, we performed a parabiosis experiment. Ncr1^{iCre}, ROSA^{mT-mG} mice were partnered with WT mice and their circulatory systems were allowed to conjoin for 20 days before m1571 g, B2m^{-/-} skin was transplanted onto the WT parabiont. At 20 days following skin transplantation, NKp46-GFP⁺ NK cells were found in the spleen and the skin graft, the majority of which were negative for anti-CD45 antibody injected I.V. NKp46-GFP⁺ NK cells migrated into the dermis and epidermis of m157^{tg},B2m^{-/-} skin followed by a large T cell infiltrate into the skin grafts. To directly test recruitment from circulation, we sorted splenic NKp46-GFP CD49b⁺CD49a⁻ cNK cells from Ncr1^{iCre}, ROSA^{mT-mG} mice and injected them I.V. into WT mice one day following m157^{tg}, B2m^{-/-} skin transplantation. After 20 days, NKp46-GFP⁺ NK cells were readily identified circulating in the recipient liver and a few NKp46-GFP⁺ NK cells were detected in the skin grafts, which had exited the circulation. Together, these data demonstrate that NK cells in the donor graft are recruited from the circulation and migrate into the dermis and epidermis where they likely adopt tissue-resident cell properties.

Next, we took genetic approaches to block trNK cell development, and thereby augment the cytotoxic function of cNK cells in the skin. In a cancer setting, enhanced NK cell cytotoxicity is accomplished through the elimination of the immunosuppressive signal from TGFβ (37), which converts cNK to trNK cells (13). WT and Ncr1^{iCre}, Tgfbr2^{fl/fl} mice were transplanted with m157^{tg}, B2m^{-/-} skin grafts; however, no rejection was observed at day 20 post-transplant. Likewise, the deletion of transcription factor Hobit, which is required for the development of liver trNK cells (38), in recipient mice did not impact m15718, B2m^{-/-} skin grafts after 20 days. Quantification of the frequency of cNK and trNK cells in the recipient liver and skin grafts revealed increased cNK and decreased trNK cells in m157^{tg}, B2m^{-/-} skin grafts in Ncr1^{iCre}, Tgfbr2^{fl/fl} and Hobit^{-/-} compared with WT recipients. Therefore, the signal(s) in the skin that restrict NK cell cytotoxicity are not superseded by exogenous NK cell-stimulating factors, restriction of immunosuppressive TGFβ signaling or prevention of cNK to trNK cell conversion.

### Example 3. m157 in the Absence of Self MHC-I Induces Robust Skin Graft Rejection

Our previous attempts to induce rejection of skin grafts through activation of the Ly49H pathway together with loss of MHC-I failed in a syngeneic transplant system. In order to preserve CD8⁺ T cell functionality while removing NK cell inhibition through the absence of self MHC-I, we generated Ncr1^{iCre}, ROSA^{mT-mG} recipient mice that were first-generation (F1) progeny of B6×BALB/c parents and transplanted them with WT and m157^{tg} B6 skin grafts. In this setting, CD8⁺ T cells were still functional through matched MHC-I signaling while NK cells licensed by BALB/c MHC-I lost MHC-I-mediated inhibition against B6 donor skin cells. Although WT skin grafts remained intact in F1 recipients, the majority of m157^{tg} skin grafts were rejected by day 20. The remainder of m157^{tg} grafts were fully rejected by day 60 post-transplant. m157^{tg} skin graft size was significantly reduced compared with WT grafts starting from day 16 post-transplant, similar to the timeline of mOVA^{tg} skin graft rejection in WT B6 mice. Thus, an NK cell-specific activating ligand in the context of missing-self in F1 recipients induces skin graft rejection; however, this rejection does not initiate within a 48-72-hour window expected of NK cell direct cytotoxicity. Since BALB/c mice do not express Ly49H, the proportion of Ly49H-expressing cNK cells in the B6×BALB/c F1 mice was reduced compared with B6 mice. However, similar to B6 recipient mice (FIG. 2G), the majority of cNK cells that migrated into the m157^{tg} skin graft in B6×BALB/c F1 mice were Ly49H⁺ that downregulated Ly49H receptor expression upon m157 engagement in the donor skin. This data indicates that despite fewer circulating Ly49H⁻ cNK cells in B6×BALB/c F1 mice, these cells were fully active against m157-expressing B6 skin graft.

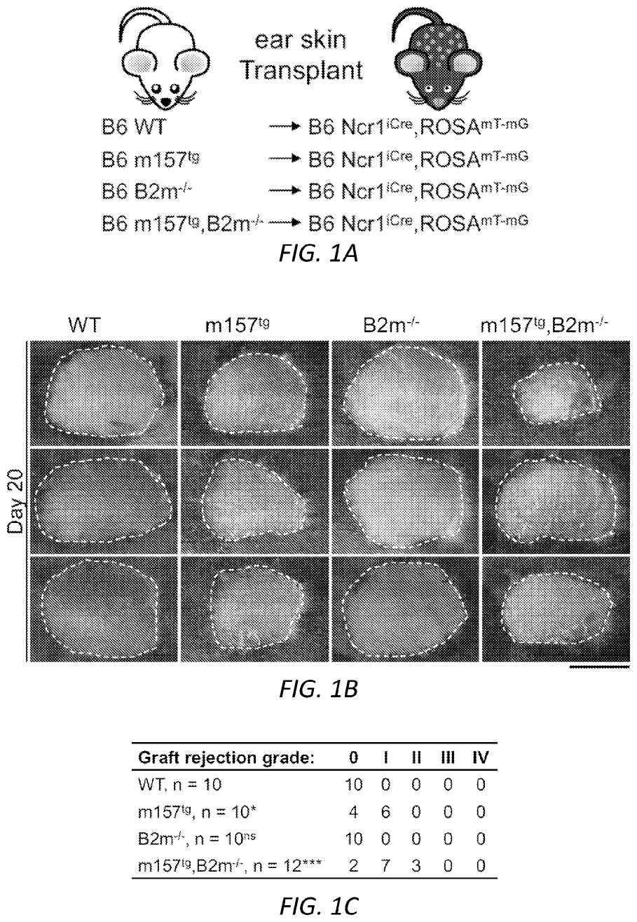

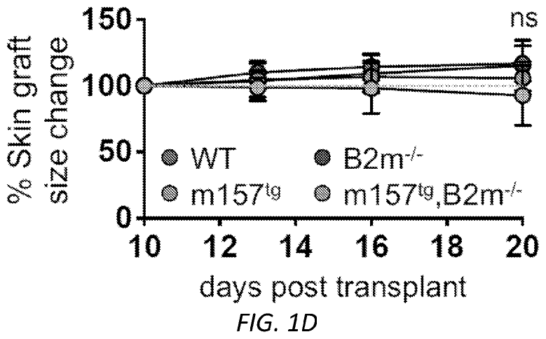

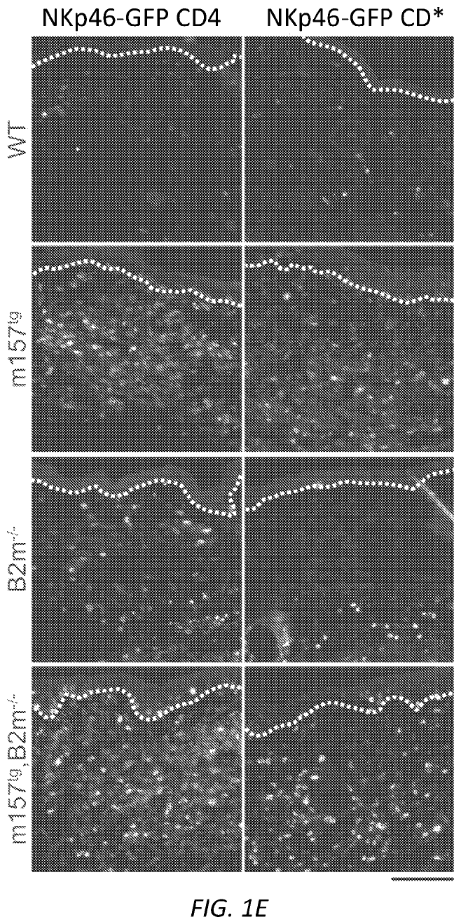

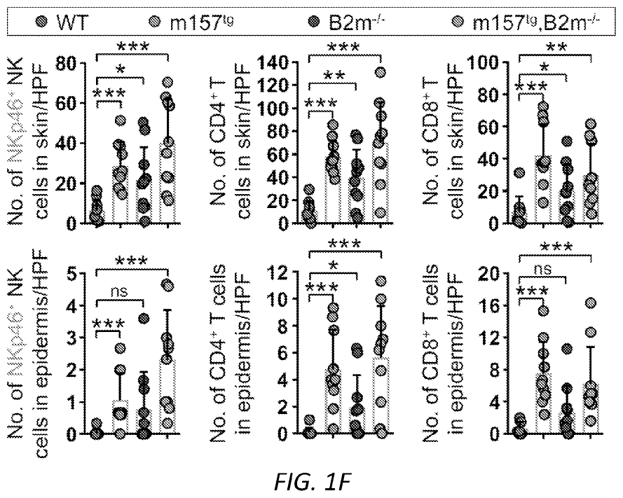

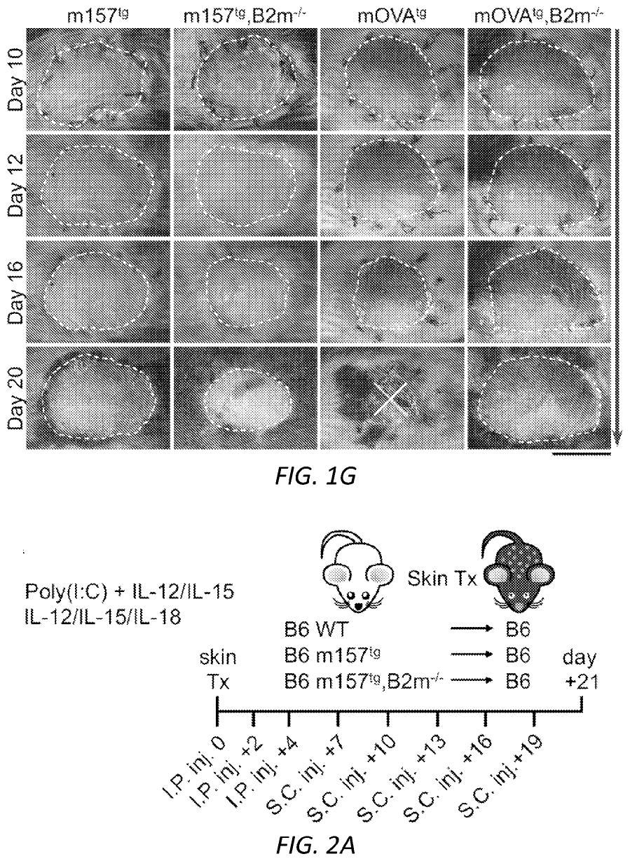

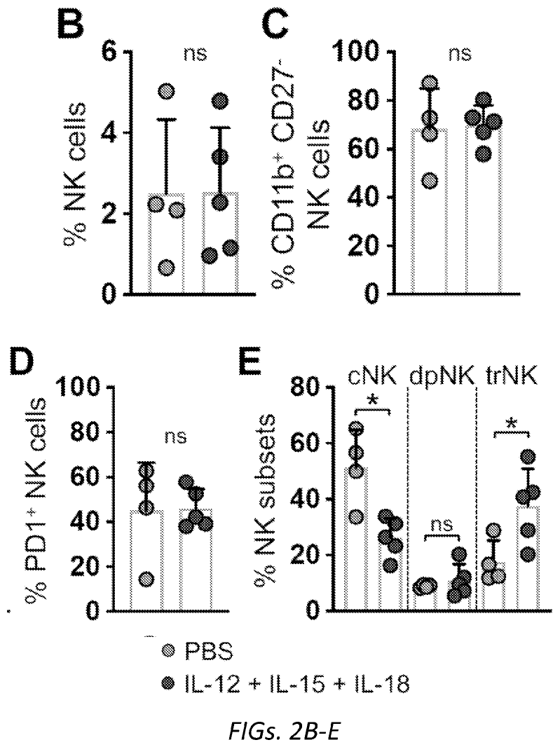

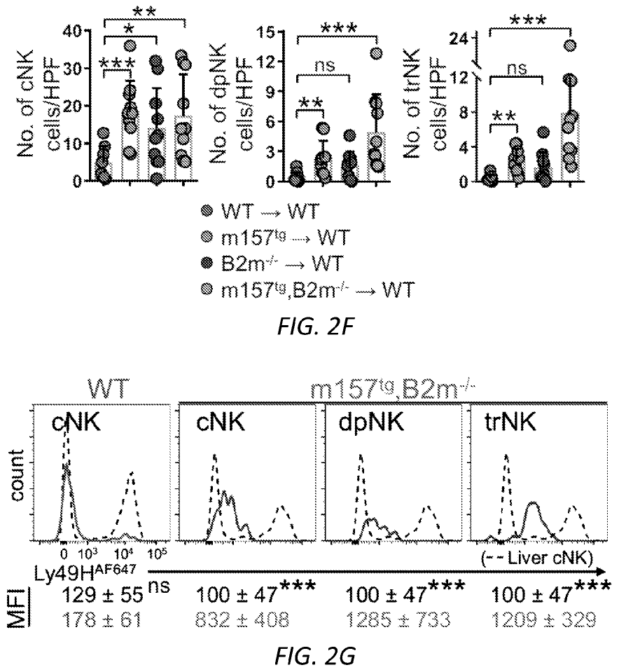

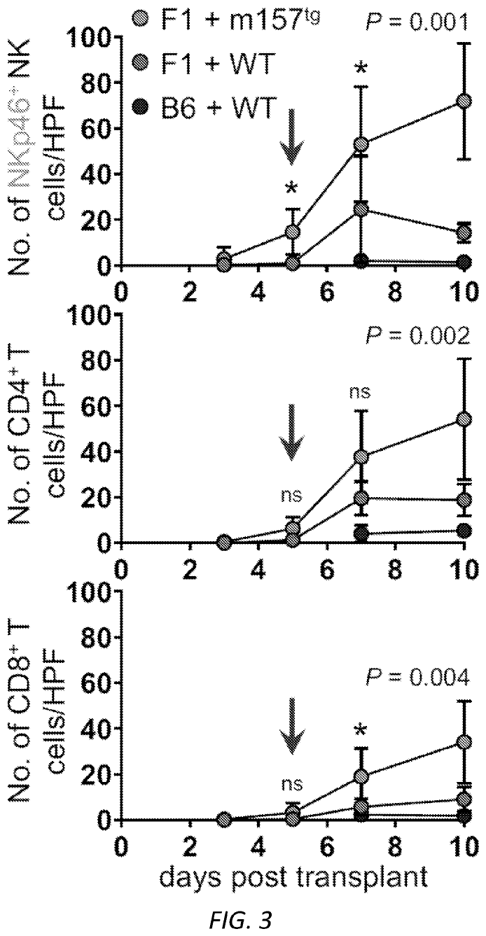

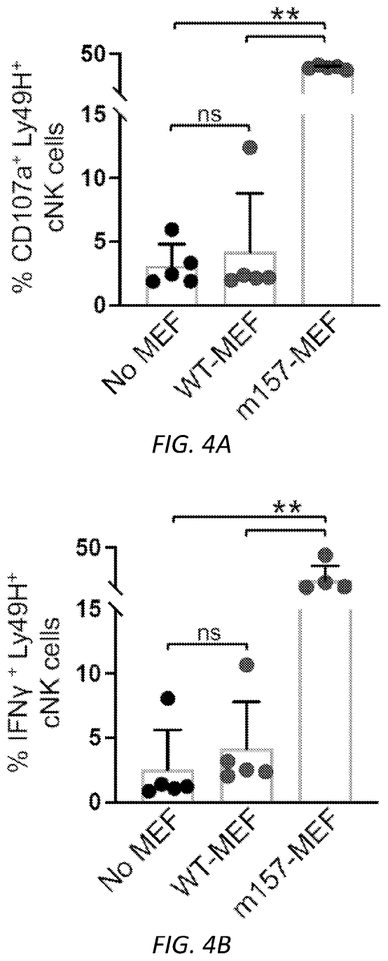

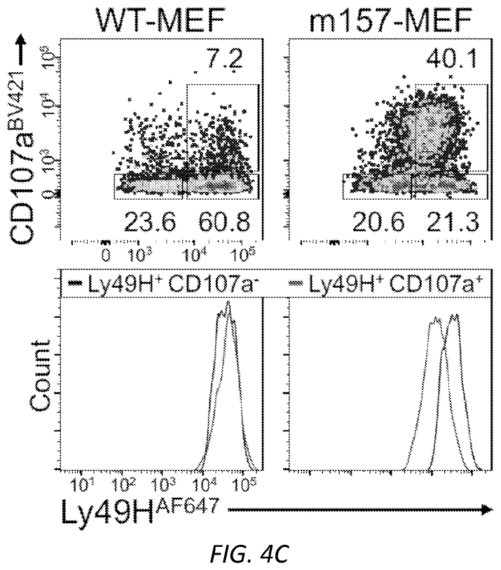

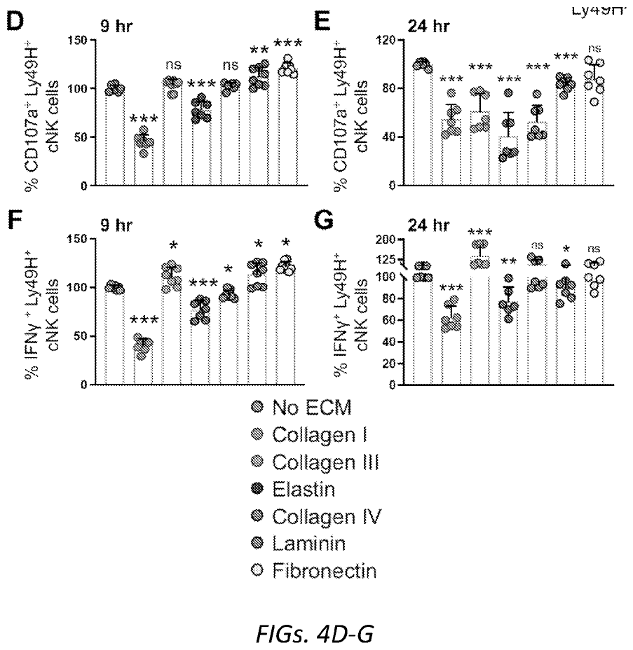

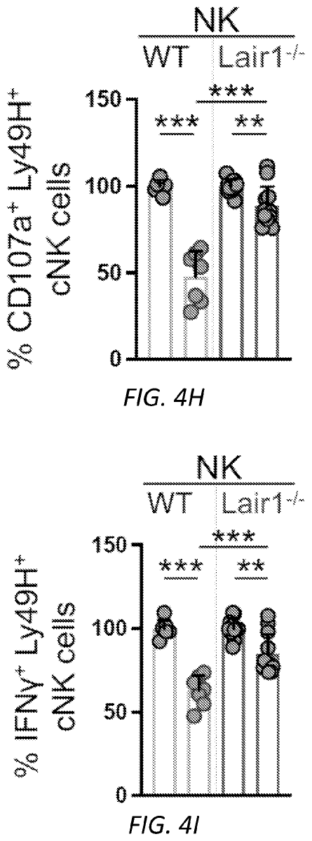

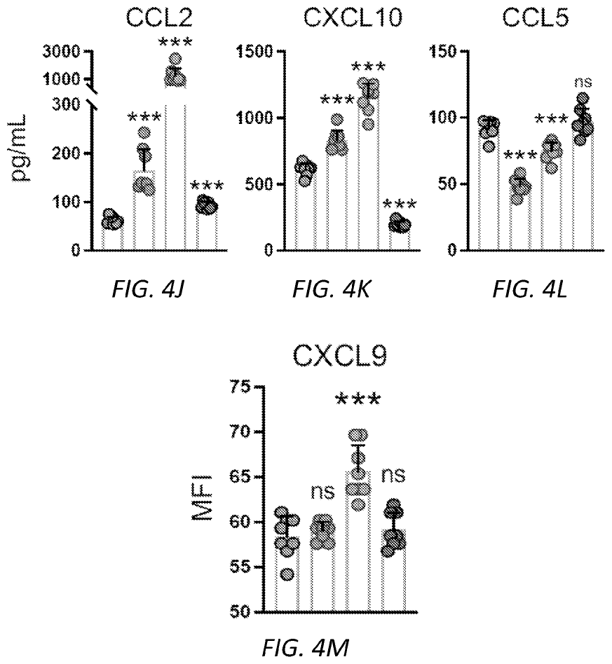

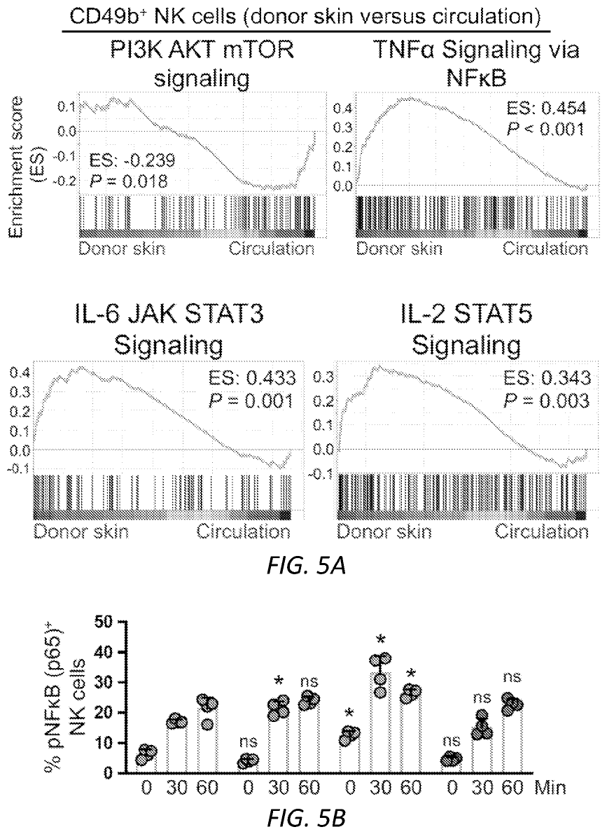

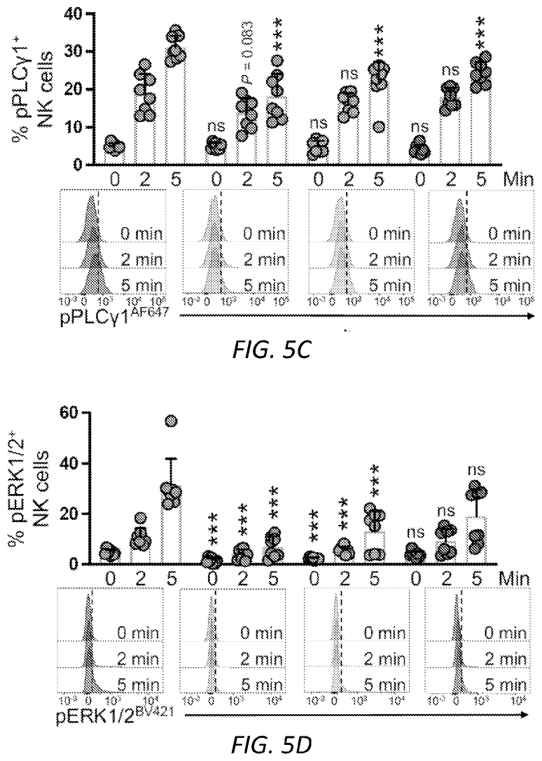

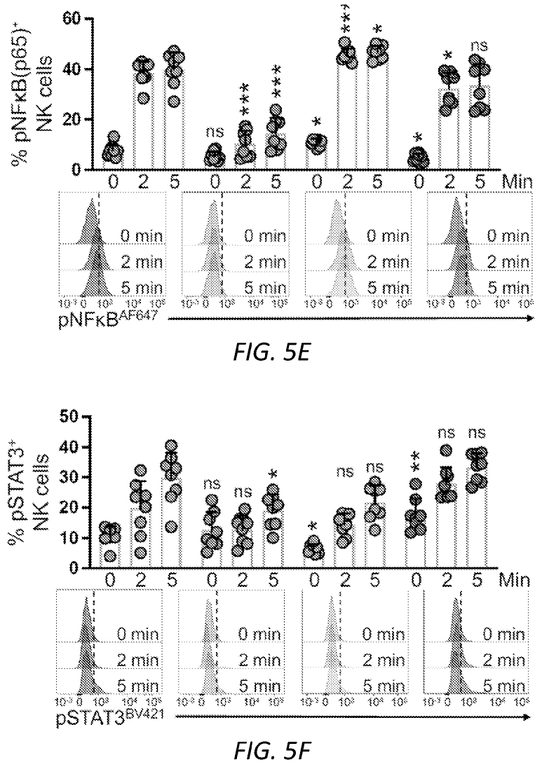

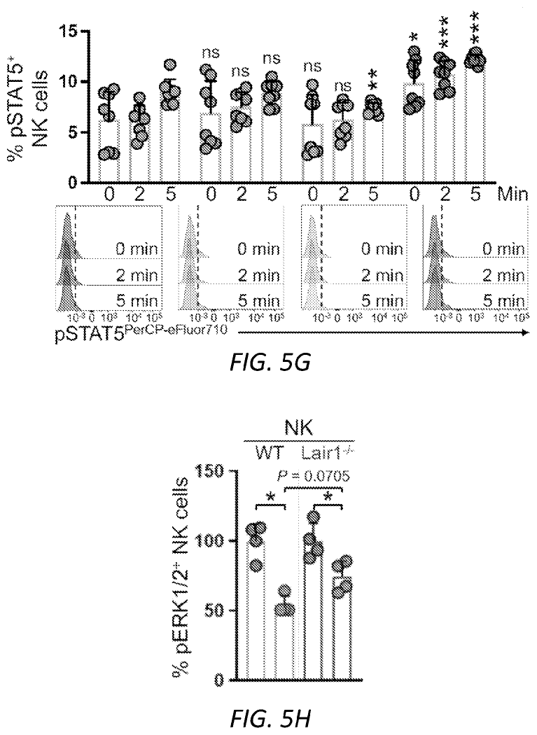

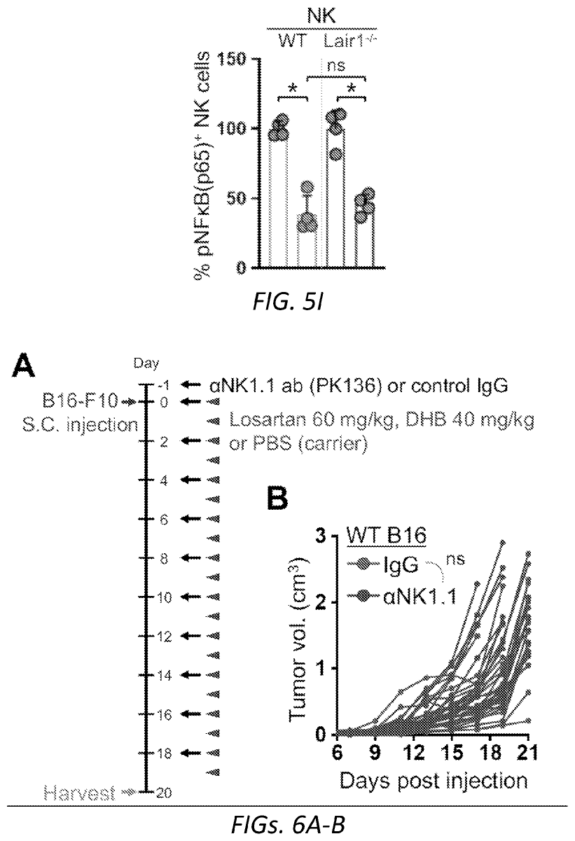

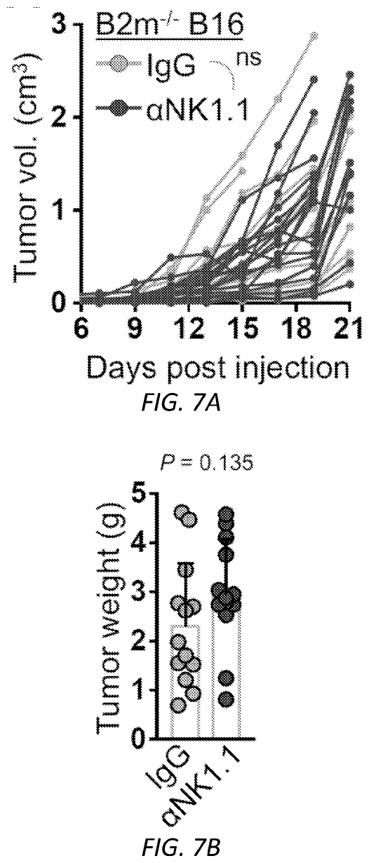

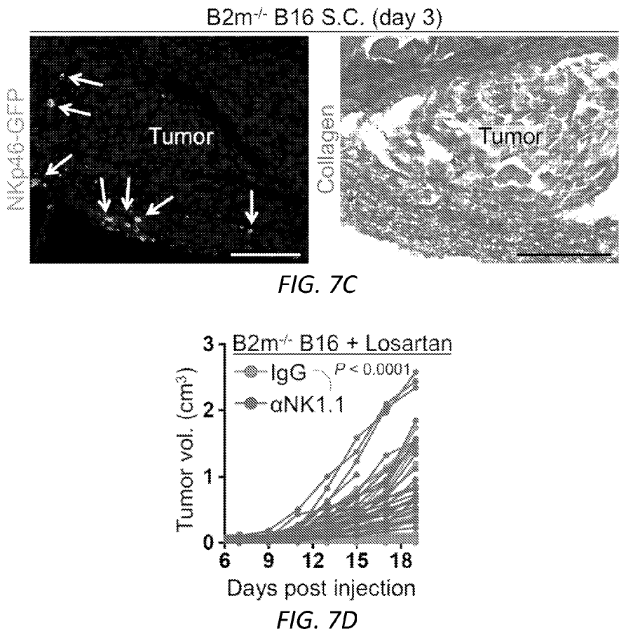

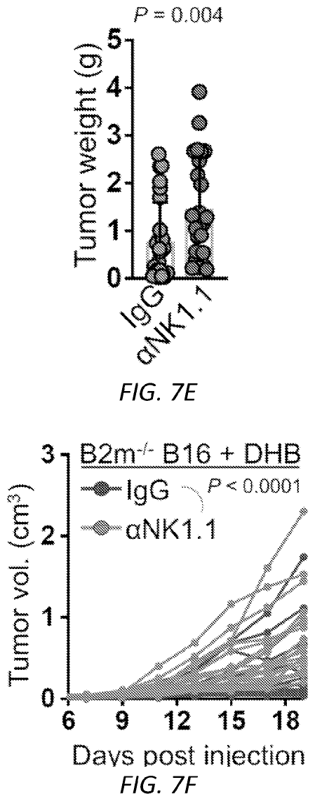

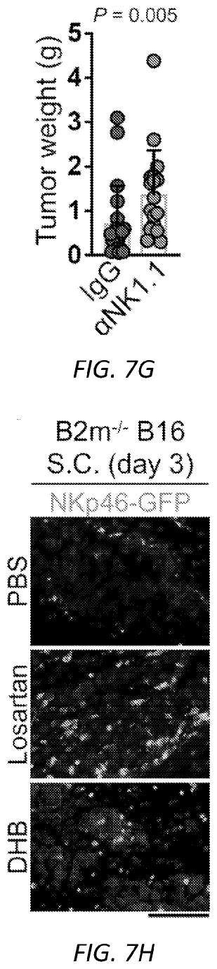

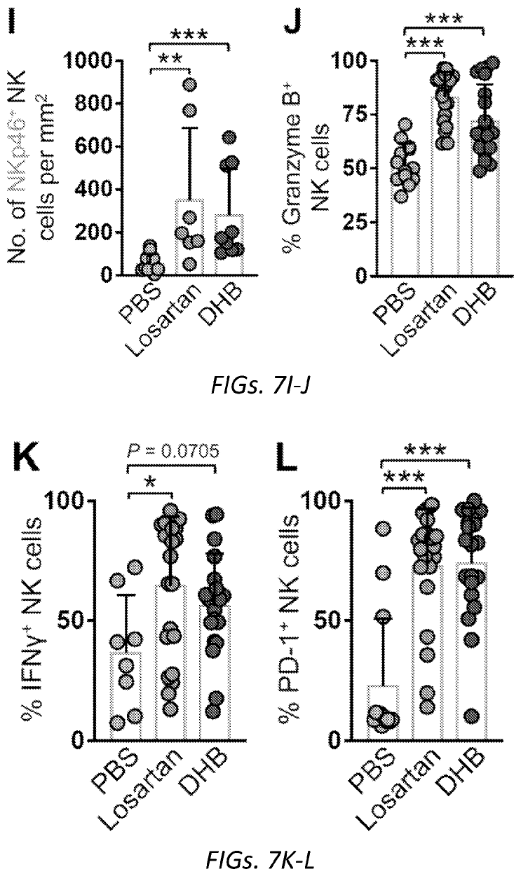

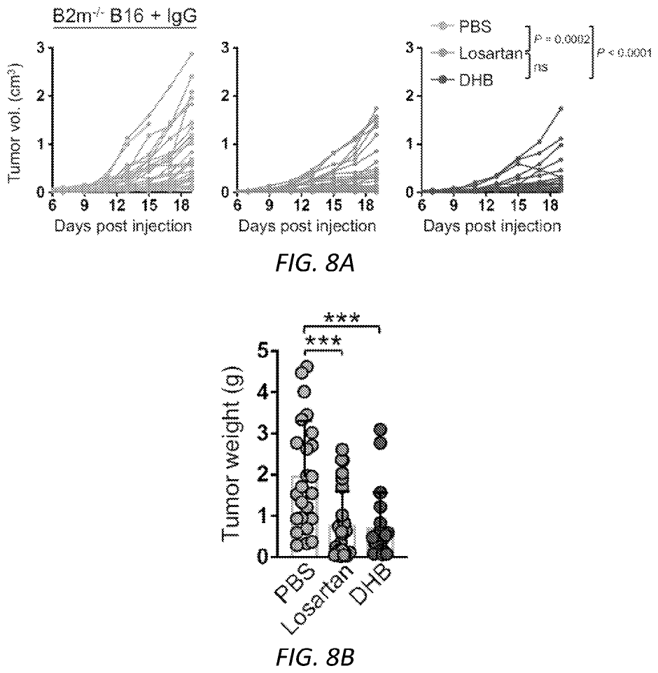

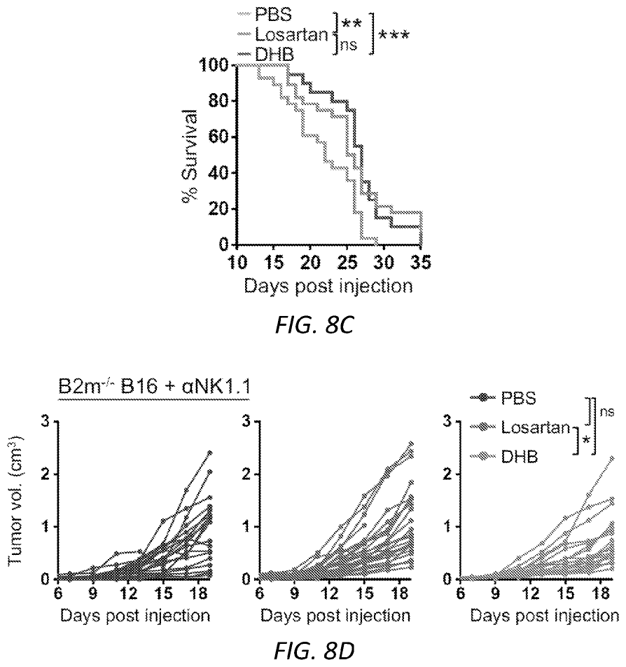

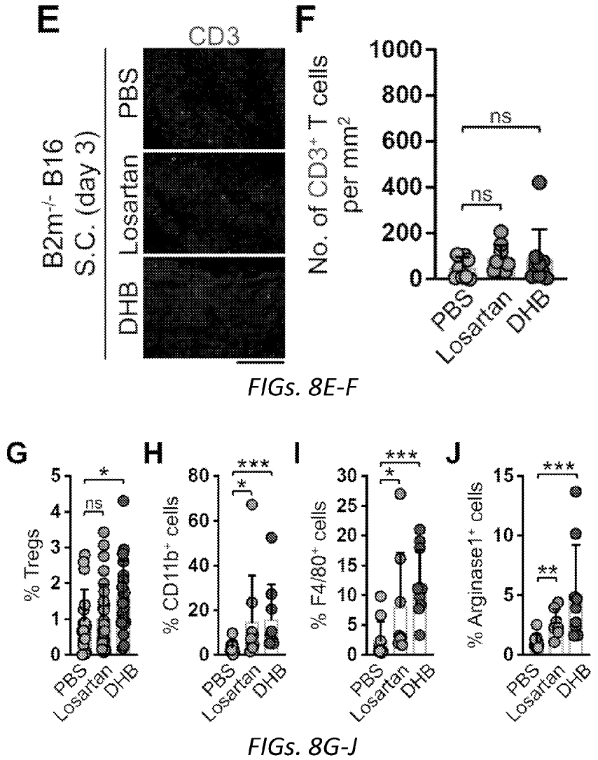

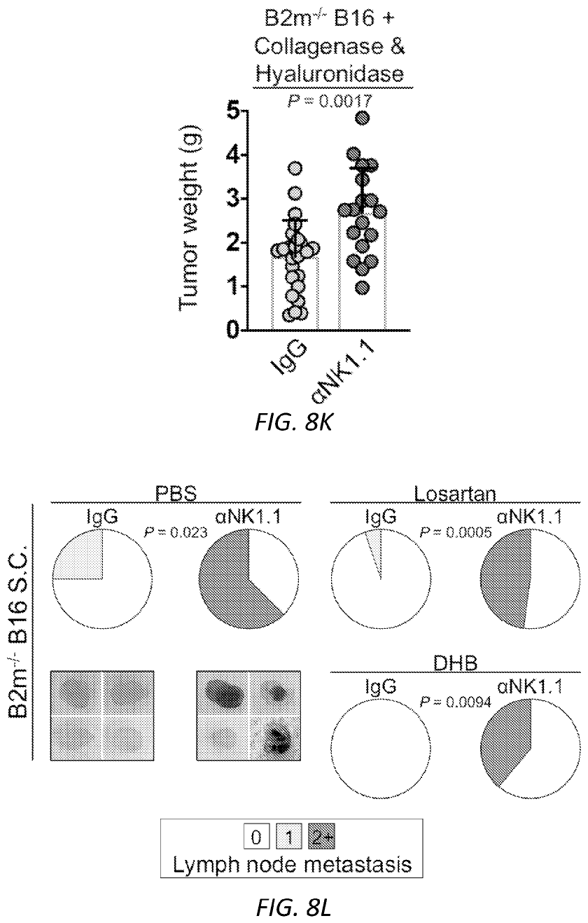

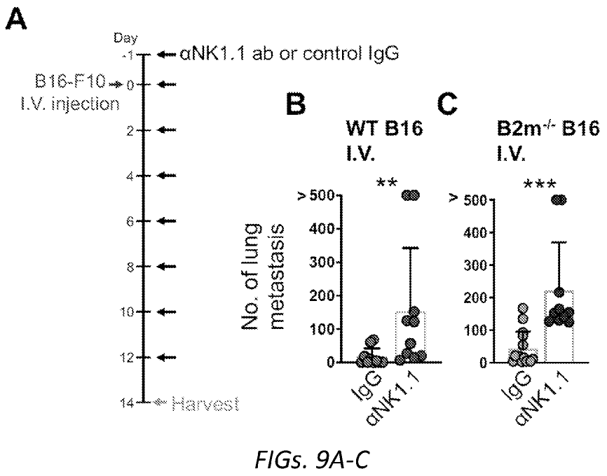

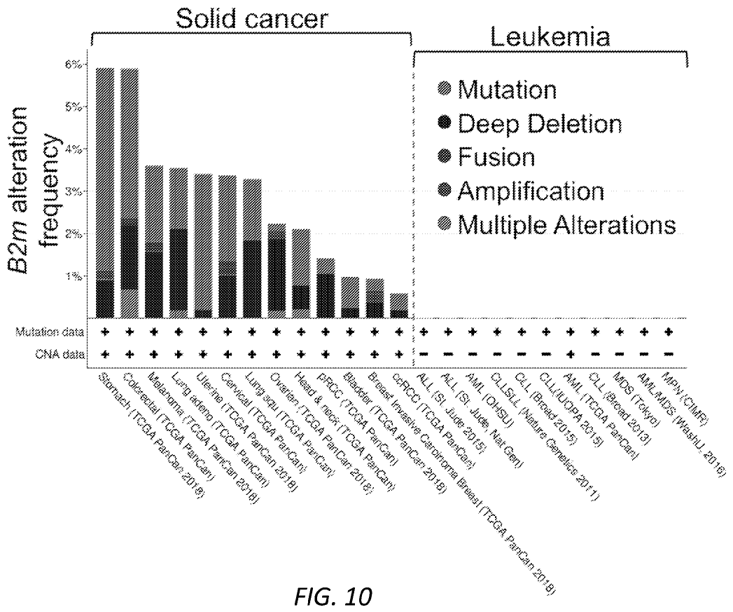

Significant increases in the number of NKp46-GFP⁺ NK, CD4⁺ T and CD8 T cells were found in m157^{tg} skin grafts at day 10 post-transplant. Quantifying the immune cell infiltrates over time revealed that significant numbers of NKp46-GFP⁺ NK cells appeared in m157^{tg} skin grafts at day 5 post-transplant while CD4⁺ T and CD8⁺ T cells infiltrated m157^{tg} skin grafts with a delay (FIG. 3). Recruitment of NK and T cells into the donor epidermis was also detected at later time points. To determine whether m157^{tg} graft rejection in F1 recipients was NK cell-dependent, we transplanted m157 g skin onto Ncr1^{iCre}, ROSA^{DTR} F1 recipient mice and treated them with diphtheria toxin (DT) and anti-NK1.1 antibody over 18 days to deplete NK cells. ROSA^{DTR} F1 recipient mice administered with DT and control antibody were used as controls. Depletion of NK cells in F1 recipients prevented rejection of m157^{tg} skin grafts. Importantly, depletion of T cells using CD4^{Cre}, ROSA^{DTR} F1 mice together with DT and CD4 and CD8 depleting antibodies also prevented rejection of m157^{tg} skin grafts. Tracking the survival of m157^{tg} skin grafts in Ncr1^{iCre}, ROSA^{DTR} mice after the termination of DT plus anti-NK1.1 antibody treatment demonstrated that recovery of NK cells in recipient mice led to complete graft rejection by day 60 post-transplant. Indeed, m157^{tg} skin grafts began to recede and develop scabs approximately 20 days after NK cell depletion was stopped. These data demonstrate that NK and T cells are required for the rejection of m157^{tg} skin grafts in the context of missing-self. The NK cell-specific nature of the m157 activation signal and the timeline of NK, and later, T cell infiltration into the skin suggests that activated NK cells may have recruited T cells as effectors to reject the skin graft.

### Example 4. cNK Cells Switch their Function from Cytotoxicity to Chemokine/Cytokine Production as Soon as they Exit the Circulation

To define the function of NK cells that infiltrated m157-expressing skin grafts, we examined the transcriptional profiles of cNK cells in the circulation (spleen and blood), cNK cells recently emigrated from the circulation into m157-expressing skin grafts, dpNK and trNK cells in the skin grafts and trNK cells in the recipient skin. This strategy enabled us to identify pathways that were up or downregulated in cNK cells early during their entry into the skin microenvironment. Strikingly, we discovered a dramatic switch in cNK cells' profile as they entered the skin grafts, which was highlighted by downregulation of cytotoxicity-related genes and upregulation of chemokines and inflammatory cytokines. Ccl1, Ccl2, Cxcl2, Cxcl9 and Cxcl10 were significantly upregulated in cNK cells in the skin graft compared to cNK cells in circulation. Likewise, Il1a, Il1b and Tnf were upregulated in donor skin cNK cells. No upregulation was seen in Ifng, Gzma or Gzmb expression, and Prf1 (perforin), Klrk1, Ccl5, Eomes and Thx21 (T-bet) were significantly downregulated in donor skin cNK cells compared with their baseline expression in circulating cNK cells. Based on RNA-Seq data and confirmed by flow cytometry, we identified TGFβRII, IL-4Rα, CCR7 and TIGIT as novel markers to distinguish cNK cells in the skin from circulating cNK cells. Together, these data demonstrate that upon entry into the skin, cNK cells undergo a drastic change in their function, which involves downregulating their cytotoxic program while boosting their ability to provide ‘help’ to neighboring immune cell populations through the production of chemokines and inflammatory cytokines.

Considering the significant upregulation of Tigit, Pdcd1 and Ctla4 in cNK cells entering the skin grafts, we examined the impact of immune checkpoint blockade on the rejection of m157-expressing skin grafts. PD-1, TIGIT or TIGIT/PD-1/CTLA4 triple antibody blockade did not result in rejection of m157^{tg} or m157^{tg},B2m^{-/-} skin grafts in WT B6 recipients. Further, TIGIT/PD-1/CTLA4 triple antibody blockade did not accelerate the rejection of m157^{tg} skin grafts in F1 recipients, indicating the persistent lack of NK cell direct cytotoxicity in the skin grafts.

### Example 5. ECM Proteins as Potential Modulators of cNK Cell's Effector Functions in the Skin

To identify the mediator(s) of NK cell functional switch in the skin, we examined the components of the skin microenvironment that interacted with NK cells as soon as they entered the skin grafts at day 5 post-transplant. NK cells infiltrated broadly into m157^{tg} dermal ECM, surrounded by collagen and to a lesser extent elastin. In addition, occasional NK cells were detected contacting dermal fibroblasts at day 5 post-transplant. To elucidate the impact of ECM proteins and fibroblasts on cNK cell cytotoxicity, we examined splenic cNK cell degranulation and cytokine production in co-culture assays. WT mouse embryonic fibroblasts (WT-MEF) did not block Ly49H⁺ cNK cell degranulation or IFNγ production in response to IL-12 and IL-15 stimulation (FIGS. 4A and B). Furthermore, fibroblasts expressing m157 (m157-MEF) markedly induced Ly49H⁺ cNK cell degranulation and IFNγ production, which was associated with Ly49H downregulation (FIGS. 4A-C). In contrast, major dermal ECM proteins, collagen I, III and elastin, potently blocked Ly49H⁺ cNK cell degranulation after 9 and 24 hours co-culture with m157-MEF (FIGS. 4D and E). Collagen I and elastin also suppressed IFNγ production by Ly49H⁺ cNK cells exposed to m157-MEF (FIGS. 4F and G). On the other hand, major epidermal basement membrane proteins, collagen IV, laminin and fibronectin, played a minor role in altering NK cell functions (FIGS. 4D-G). Leukocyte Associated Immunoglobulin Like Receptor 1 (LAIR1) is an inhibitory receptor expressed by NK cells that binds collagens (39, 40). Deletion of Lair/partially rescued collagen I-induced blockade of Ly49H⁻ cNK cell degranulation and IFNγ production upon in vitro exposure to m157-MEF (FIGS. 4H and I). However, global Lair1 deletion failed to induce the rejection of m157^{tg}, B2m^{-/-} skin graft in Lair1^{-/-} mice. This suggests that the partial LAIR 1 dependency observed in our in vitro study may be compensated by complex interplay between NK cells and other ECM proteins in the skin. The close linkage between Ncr1 and Lair I genes on chromosome 7 in mice prevented us from generating Ncr1^{iCre}, Lair1^{fl/fl} mice to examine the NK cell-specific role of LAIR1 in vivo. Strikingly, Collagen I and III significantly induced CCL2 and CXCL10 but suppressed CCL5 release by splenic cNK cells cultured with m157-MEF (FIGS. 4J-L). Collagen III also induced CXCL9 release by cNK cells (FIG. 4M).

To gain a greater insight into the underlying molecular mechanisms linking the ECM to changed NK cell functionalities, we investigated the signaling pathways that were associated with the switch in cNK cells' profile from cytotoxicity to an inflammatory response. cNK cells entering the skin downregulated PI3K-AKT pathway while upregulating NFκB, STAT3 and STAT5 signaling pathways compared with circulating cNK cells (FIG. 5A). Interestingly, we discovered that Collagen III significantly induced phospho-NFκB (p65) in splenic cNK cells at baseline, which was highly amplified after 30- and 60-minutes exposure to m157-MEF (FIG. 5B). To examine the impact of ECM proteins on NK cell signaling upon activation in a time-controlled setting that excluded the possibility for ECM proteins to physically block activating ligand-receptor interactions, we activated NK cells with H₂O₂ in the presence of collagens and elastin (41). Notably, collagens and elastin markedly reduced cytotoxicity-associated pPLCγ1 and pERK1/2 levels in the NK cells (FIGS. 5C and D). In contrast, collagen III increased pNFκB in NK cells at baseline and after 2- and 5-minute exposure to H₂O₂ (FIG. 5E). Interestingly, elastin increased pSTAT3 at baseline and highly induced pSTAT5 at baseline and after 2- and 5-minutes exposure to H₂O₂ (FIGS. 5F and G). Collagen I showed a suppressive effect on pNFκB and pSTAT3 (FIGS. 5E and F). Loss of Lair I in NK cells partially reversed pERK1/2 suppression by collagen I but had no impact on NFκB phosphorylation after 2 minutes exposure to H₂O₂ (FIGS. 5H and I). Consistent with their critical role in regulating cNK cell function, we found the expression of multiple ECM protein receptors on cNK cells entering the skin microenvironment. These findings indicate that ECM proteins including collagen I, III and elastin are potent regulators of cNK cell function, which collectively may lead to reduced cytotoxicity and increased chemokine and cytokine production by NK cells as they enter the peripheral tissues.

### Example 6. ECM Proteins Suppress NK Cell Antitumor Response in the Skin

Considering the essential role of ECM proteins in skin homeostasis and wound healing, our attempts to remove or degrade ECM proteins in the skin transplantation system led to the failure of skin engraftment. Thus, we examined the role of ECM proteins in suppressing NK cell cytotoxicity against cancer cells in the skin. We subcutaneously (S.C.) injected B2m^{-/-} B16-F10 melanoma cells (B16) into the flanks of WT mice and treated them with control IgG or anti-NK1.1 antibody to determine if NK cells inhibited the growth of MHC-I-deficient tumors in the skin (FIG. 6A). Similar to WT melanoma, NK cell depletion did not impact the growth or terminal weight of B2m^{-/-} melanoma in the skin (FIGS. 7A and B, and FIG. 6B). NK cells infiltrated B2m^{-/-} melanoma as early as day 3 post-tumor injection but were embedded in a collagen-rich ECM surrounding the tumor foci (FIG. 7C). To investigate the role of tumor collagen deposition in suppressing NK cell immunity against melanoma in the skin, we treated mice that received B2m^{-/-} B16 S.C. with losartan (60 mg/kg) to block collagen synthesis or collagen prolyl 4-hydroxylase inhibitor, 3,4-dihydroxybenzoic acid (DHB, 40 mg/kg), which blocks collagen assembly into a triple helix (FIG. 6A) (42, 43). Importantly, losartan and DHB treatment protected the mice from B2m^{-/-} melanoma growth in the skin in an NK cell-dependent manner, which led to a significant survival benefit (FIGS. 7D-G and FIGS. 8A-D). NK cells highly infiltrated the tumor parenchyma in losartan and DHB-treated mice and showed markedly elevated expression of granzyme B, IFNγ and PD-1 (FIGS. 8H-L). Unlike NK cells, Losartan and DHB treatment did not induce T cell recruitment into the B2m^{-/-} B16 tumors (FIGS. 8E and F). Furthermore, the proportions of tumor infiltrating regulatory T cells (Tregs), macrophages and myeloid derived suppressor cells (MDSCs) remained unaltered or increased following Losartan and DHB treatments (FIGS. 8G-J). To further confirm that the NK cell immunity unleashed upon losartan and DHB treatment was related to their effects on blocking collagen deposition in the tumor, we treated a group of mice that received B2m^{-/-} B16 S.C. with collagenase and hyaluronidase mixture and found a similar dependency of tumor growth on NK cells (FIG. 8K). Interestingly, NK cell depletion in PBS (carrier control), losartan and DHB-treated mice resulted in lymph node metastasis of B2m^{-/-} melanoma, suggesting a role for NK cells in the clearance of metastasizing melanoma cells (FIG. 8L). Consistent with this effect, and in contrast to the lack of NK cell's ability to control melanoma in the skin, NK cells effectively killed intravenously (I. V.) injected WT and B2m^{-/-} B16 cells and suppressed melanoma lung metastasis (FIGS. 9A-C). Collectively, these data indicate strong NK cell killing of MHC-I-deficient tumor cells in the circulation, which is likely blocked by ECM proteins in peripheral tissues.

To translate our fundamental findings to human cancers, we analyzed the TCGA and other publicly available datasets for the frequency of B2M gene alteration and mutations in multiple types of solid cancers and leukemias. While B2M gene mutations and deletion were found across solid cancer types including those with low mutational burden, leukemias did not carry any aberrations in the B2M gene (FIG. 10). Furthermore, B2M gene expression in acute myeloid leukemia (AML) samples was consistently high and showed the least variance compared with solid cancer types. These findings indicate a remarkable resistance to downregulation of the B2M gene in blood cancers even when confronted with CD8⁺ T cell pressure. Thus, circulating cytotoxic NK cells likely eliminate leukemias that lose MHC-I while solid cancers that downregulate MHC-I in peripheral tissues are protected due to the impaired NK cell cytotoxicity caused by ECM proteins.

### REFERENCES

1. F. Garrido, N. Aptsiauri, Cancer immune escape: MHC expression in primary tumours versus metastases. Immunology 158, 255-266 (2019).
2. S. Bessoles, C. Grandclement, E. Alari-Pahissa, J. Gehrig, B. Jeevan-Raj, W. Held, Adaptations of Natural Killer Cells to Self-MHC Class I. Front Immunol 5, 349 (2014).
3 E. O. Long, H. S. Kim, D. Liu, M. E. Peterson, S. Rajagopalan, Controlling natural killer cell responses: integration of signals for activation and inhibition. Annu Rev Immunol 31, 227-258 (2013).
4. F. M. Karlhofer, R. K. Ribaudo, W. M. Yokoyama, MHC class I alloantigen specificity of Ly-49+IL-2-activated natural killer cells. Nature 358, 66-70 (1992).
5. M. Bennett, Y. Y. Yu, E. Stoneman, R. M. Rembecki, P. A. Mathew, K. F. Lindahl, V. Kumar, Hybrid resistance: ‘negative’ and ‘positive’ signaling of murine natural killer cells. Semin Immunol 7, 121-127 (1995).
6. V. Kumar, T. George, Y. Y. Yu, J. Liu, M. Bennett, Role of murine NK cells and their receptors in hybrid resistance. Curr Opin Immunol 9, 52-56 (1997).
7. J. O. Manilay, M. Sykes, Natural killer cells and their role in graft rejection. Current opinion in immunology 10, 532-538 (1998).
8 M. Zijlstra, H. Auchincloss, Jr., J. M. Loring, C. M. Chase, P. S. Russell, R. Jaenisch, Skin graft rejection by beta 2-microglobulin-deficient mice. The Journal of experimental medicine 175, 885-893 (1992).
9. L. Riggan, A. G. Freud, T. E. O'Sullivan, True Detective: Unraveling Group 1 Innate Lymphocyte Heterogeneity. Trends Immunol 40, 909-921 (2019).
10. D. K. Sojka, Z. Tian, W. M. Yokoyama, Tissue-resident natural killer cells and their potential diversity. Semin Immunol 26, 127-131 (2014).
11. C. Seillet, G. T. Belz, Differentiation and diversity of subsets in group 1 innate lymphoid cells. Int Immunol 28, 3-11 (2016).
12. V. S. Cortez, T. K. Ulland, L. Cervantes-Barragan, J. K. Bando, M. L. Robinette, Q. Wang, A. J. White, S. Gilfillan, M. Cella, M. Colonna, SMAD4 impedes the conversion of NK cells into ILC1-like cells by curtailing non-canonical TGF-beta signaling. Nat Immunol 18, 995-1003 (2017).
13. Y. Gao, F. Souza-Fonseca-Guimaraes, T. Bald, S. S. Ng, A. Young, S. F. Ngiow, J. Rautela, J. Straube, N. Waddell, S. J. Blake, J. Yan, L. Bartholin, J. S. Lee, E. Vivier, K. Takeda, M. Messaoudene, L. Zitvogel, M. W. L. Teng, G. T. Belz, C. R. Engwerda, N. D. Huntington, K. Nakamura, M. Holzel, M. J. Smyth, Tumor immunoevasion by the conversion of effector NK cells into type 1 innate lymphoid cells. Nat Immunol 18, 1004-1015 (2017).
14. L. L. Lanier, NK cell recognition. Annu Rev Immunol 23, 225-274 (2005).
15. M. D. Bern, B. A. Parikh, L. Yang, D. L. Beckman, J. Poursine-Laurent, W. M. Yokoyama, Inducible down-regulation of MHC class I results in natural killer cell tolerance. The Journal of experimental medicine 216, 99-116 (2019).
16. W. M. Yokoyama, S. Kim, How do natural killer cells find self to achieve tolerance? Immunity 24, 249-257 (2006).
17. P. Brodin, K. Karre, P. Hoglund, NK cell education: not an on-off switch but a tunable rheostat. Trends Immunol 30, 143-149 (2009).
18. J. M. Elliott, W. M. Yokoyama, Unifying concepts of MHC-dependent natural killer cell education. Trends Immunol 32, 364-372 (2011).
19. J. S. Orange, Formation and function of the lytic NK-cell immunological synapse. Nat Rev Immunol 8, 713-725 (2008).
20. R. P. Wallin, V. Screpanti, J. Michaelsson, A. Grandien, H. G. Ljunggren, Regulation of perforin-independent NK cell-mediated cytotoxicity. Eur J Immunol 33, 2727-2735 (2003).
21. R. J. Lebbink, L. Meyaard, Non-MHC ligands for inhibitory immune receptors: novel insights and implications for immune regulation. Mol Immunol 44, 2153-2164 (2007).
22. S. Bauer, V. Groh, J. Wu, A. Steinle, J. H. Phillips, L. L. Lanier, T. Spies, Activation of NK cells and T cells by NKG2D, a receptor for stress-inducible MICA. Science 285, 727-729 (1999).
23. H. R. Smith, J. W. Heusel, I. K. Mehta, S. Kim, B. G. Dorner, O. V. Naidenko, K. Iizuka, H. Furukawa, D. L. Beckman, J. T. Pingel, A. A. Scalzo, D. H. Fremont, W. M. Yokoyama, Recognition of a virus-encoded ligand by a natural killer cell activation receptor. Proceedings of the National Academy of Sciences of the United States of America 99, 8826-8831 (2002).
24. Q. Hammer, T. Ruckert, C. Romagnani, Natural killer cell specificity for viral infections. Nat Immunol 19, 800-808 (2018).
25. K. Karre, H. G. Ljunggren, G. Piontek, R. Kiessling, Selective rejection of H-2-deficient lymphoma variants suggests alternative immune defence strategy. Nature 319, 675-678 (1986).
26. E. Narni-Mancinelli, J. Chaix, A. Fenis, Y. M. Kerdiles, N. Yessaad, A. Reynders, C. Gregoire, H. Luche, S. Ugolini, E. Tomasello, T. Walzer, E. Vivier, Fate mapping analysis of lymphoid cells expressing the NKp46 cell surface receptor. Proceedings of the National Academy of Sciences of the United States of America 108, 18324-18329 (2011).
27. S. K. Tripathy, P. A. Keyel, L. Yang, J. T. Pingel, T. P. Cheng, A. Schneeberger, W. M. Yokoyama, Continuous engagement of a self-specific activation receptor induces NK cell tolerance. The Journal of experimental medicine 205, 1829-1841 (2008).
28. J. M. Elliott, J. A. Wahle, W. M. Yokoyama, MHC class I-deficient natural killer cells acquire a licensed phenotype after transfer into an MHC class I-sufficient environment. The Journal of experimental medicine 207, 2073-2079 (2010).
29. N. T. Joncker, N. Shifrin, F. Delebecque, D. H. Raulet, Mature natural killer cells reset their responsiveness when exposed to an altered MHC environment. The Journal of experimental medicine 207, 2065-2072 (2010).
30. N. D. Huntington, The unconventional expression of IL-15 and its role in NK cell homeostasis. Immunol Cell Biol 92, 210-213 (2014).
31. A. Marcais, S. Viel, M. Grau, T. Henry, J. Marvel, T. Walzer, Regulation of mouse NK cell development and function by cytokines. Front Immunol 4, 450 (2013).
32. T. Hydes, A. Noll, G. Salinas-Riester, M. Abuhilal, T. Armstrong, Z. Hamady, J. Primrose, A. Takhar, L. Walter, S. I. Khakoo, IL-12 and IL-15 induce the expression of CXCR6 and CD49a on peripheral natural killer cells. Immunity, inflammation and disease 6, 34-46 (2018).
33. X. Ni, B. Fu, J. Zhang, R. Sun, Z. Tian, H. Wei, Cytokine-Based Generation of CD49a(+)Eomes(−/+) Natural Killer Cell Subsets. Frontiers in immunology 9, 2126 (2018).
34. V. S. Cortez, L. Cervantes-Barragan, M. L. Robinette, J. K. Bando, Y. Wang, T. L. Geiger, S. Gilfillan, A. Fuchs, E. Vivier, J. C. Sun, M. Cella, M. Colonna, Transforming Growth Factor-beta Signaling Guides the Differentiation of Innate Lymphoid Cells in Salivary Glands. Immunity 44, 1127-1139 (2016).
35. L. Tang, H. Peng, J. Zhou, Y. Chen, H. Wei, R. Sun, W. M. Yokoyama, Z. Tian, Differential phenotypic and functional properties of liver-resident NK cells and mucosal ILC1s. J Autoimmun 67, 29-35 (2016).
36. D. K. Sojka, B. Plougastel-Douglas, L. Yang, M. A. Pak-Wittel, M. N. Artyomov, Y. Ivanova, C. Zhong, J. M. Chase, P. B. Rothman, J. Yu, J. K. Riley, J. Zhu, Z. Tian, W. M. Yokoyama, Tissue-resident natural killer (NK) cells are cell lineages distinct from thymic and conventional splenic NK cells. eLife 3, e01659 (2014).
37. S. Viel, A. Marcais, F. S. Guimaraes, R. Loftus, J. Rabilloud, M. Grau, S. Degouve, S. Djebali, A. Sanlaville, E. Charrier, J. Bienvenu, J. C. Marie, C. Caux, J. Marvel, L. Town, N. D. Huntington, L. Bartholin, D. Finlay, M. J. Smyth, T. Walzer, TGF-beta inhibits the activation and functions of NK cells by repressing the mTOR pathway. Sci Signal 9, ra19 (2016).
38. L. K. Mackay, M. Minnich, N. A. Kragten, Y. Liao, B. Nota, C. Seillet, A. Zaid, K. Man, S. Preston, D. Freestone, A. Braun, E. Wynne-Jones, F. M. Behr, R. Stark, D. G. Pellicci, D. I. Godfrey, G. T. Belz, M. Pellegrini, T. Gebhardt, M. Busslinger, W. Shi, F. R. Carbone, R. A. van Lier, A. Kallies, K. P. van Gisbergen, Hobit and Blimp1 instruct a universal transcriptional program of tissue residency in lymphocytes. Science (New York, N.Y 352, 459-463 (2016).
39. R. J. Lebbink, T. de Ruiter, G. J. Kaptijn, D. G. Bihan, C. A. Jansen, P. J. Lenting, L. Meyaard, Mouse leukocyte-associated Ig-like receptor-1 (mLAIR-1) functions as an inhibitory collagen-binding receptor on immune cells. Int Immunol 19, 1011-1019 (2007).
40. L. Meyaard, The inhibitory collagen receptor LAIR-1 (CD305). J Leukoc Biol 83, 799-803 (2008).
41. A. Corcoran, T. G. Cotter, Redox regulation of protein kinases. FEBS J 280, 1944-1965 (2013).
42. B. Diop-Frimpong, V. P. Chauhan, S. Krane, Y. Boucher, R. K. Jain, Losartan inhibits collagen I synthesis and improves the distribution and efficacy of nanotherapeutics in tumors. Proceedings of the National Academy of Sciences of the United States of America 108, 2909-2914 (2011).
43. G. Xiong, R. L. Stewart, J. Chen, T. Gao, T. L. Scott, L. M. Samayoa, K. O'Connor, A. N. Lane, R. Xu, Collagen prolyl 4-hydroxylase 1 is essential for HIF-1 alpha stabilization and TNBC chemoresistance. Nat Commun 9, 4456 (2018).
44. E. Narni-Mancinelli, S. Ugolini, E. Vivier, Tuning the threshold of natural killer cell responses. Curr Opin Immunol 25, 53-58 (2013).
45. F. D. Shi, H. G. Ljunggren, A. La Cava, L. Van Kaer, Organ-specific features of natural killer cells. Nat Rev Immunol 11, 658-671 (2011).
46. J. P. Bottcher, E. Bonavita, P. Chakravarty, H. Blees, M. Cabeza-Cabrerizo, S. Sammicheli, N. C. Rogers, E. Sahai, S. Zelenay, C. Reis e Sousa, NK Cells Stimulate Recruitment of cDC1 into the Tumor Microenvironment Promoting Cancer Immune Control. Cell 172, 1022-1037 e1014 (2018).
47. M. A. Degli-Esposti, M. J. Smyth, Close encounters of different kinds: dendritic cells and NK cells take centre stage. Nat Rev Immunol 5, 112-124 (2005).
48. G. Gasteiger, A. Y. Rudensky, Interactions between innate and adaptive lymphocytes. Nat Rev Immunol 14, 631-639 (2014).
49. P. Kalinski, A. Giermasz, Y. Nakamura, P. Basse, W. J. Storkus, J. M. Kirkwood, R. B. Mailliard, Helper role of NK cells during the induction of anticancer responses by dendritic cells. Mol Immunol 42, 535-539 (2005).
50. R. B. Mailliard, Y. I. Son, R. Redlinger, P. T. Coates, A. Giermasz, P. A. Morel, W. J. Storkus, P. Kalinski, Dendritic cells mediate NK cell help for Th1 and CTL responses: two-signal requirement for the induction of NK cell helper function. J Immunol 171, 2366-2373 (2003).
51. A. Martin-Fontecha, L. L. Thomsen, S. Brett, C. Gerard, M. Lipp, A. Lanzavecchia, F. Sallusto, Induced recruitment of NK cells to lymph nodes provides IFN-gamma for T(H)1 priming. Nature immunology 5, 1260-1265 (2004).
52. R. Mocikat, H. Braumuller, A. Gumy, O. Egeter, H. Ziegler, U. Reusch, A. Bubeck, J. Louis, R. Mailhammer, G. Riethmuller, U. Koszinowski, M. Rocken, Natural killer cells activated by MHC class I(low) targets prime dendritic cells to induce protective CD8 T cell responses. Immunity 19, 561-569 (2003).
53. J. P. Bottcher, E. Bonavita, P. Chakravarty, H. Blees, M. Cabeza-Cabrerizo, S. Sammicheli, N. C. Rogers, E. Sahai, S. Zelenay, E. S. C. Reis, NK Cells Stimulate Recruitment of cDC1 into the Tumor Microenvironment Promoting Cancer Immune Control. Cell, (2018).
54. A. Le Moine, M. Goldman, D. Abramowicz, Multiple pathways to allograft rejection. Transplantation 73, 1373-1381 (2002).
55. M. Wise, D. Zelenika, F. Bemelman, D. Latinne, H. Bazin, S. Cobbold, H. Waldmann, CD4 T cells can reject major histocompatibility complex class I-incompatible skin grafts. Eur J Immunol 29, 156-167 (1999).
56. J. Marino, J. Paster, G. Benichou, Allorecognition by T Lymphocytes and Allograft Rejection. Front Immunol 7, 582 (2016).
57. G. Habif, A. Crinier, P. Andre, E. Vivier, E. Narni-Mancinelli, Targeting natural killer cells in solid tumors. Cell Mol Immunol 16, 415-422 (2019).
58. G. Nayyar, Y. Chu, M. S. Cairo, Overcoming Resistance to Natural Killer Cell Based Immunotherapies for Solid Tumors. Front Oncol 9, 51 (2019).
59. M. H. Sandel, F. M. Speetjens, A. G. Menon, P. A. Albertsson, P. H. Basse, M. Hokland, J. F. Nagelkerke, R. A. Tollenaar, C. J. van de Velde, P. J. Kuppen, Natural killer cells infiltrating colorectal cancer and MHC class I expression. Mol Immunol 42, 541-546 (2005).
60. J. J. Trombetta, D. Gennert, D. Lu, R. Satija, A. K. Shalek, A. Regev, Preparation of Single-Cell RNA-Seq Libraries for Next Generation Sequencing. Curr Protoc Mol Biol 107, 4 22 21-17 (2014).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject who has a solid tumor, the method comprising administering to the subject a therapeutically effective amount of (i) blocking antibodies to ECM components and/or (ii) inhibitors of collagen deposition or production.

2. A method of treating a subject who has a viral infection, the method comprising administering to the subject a therapeutically effective amount of (i) blocking antibodies to ECM components and/or (ii) inhibitors of collagen deposition or production.

3. A method of promoting cytotoxicity of natural killer (NK) cells in a subject, the method comprising administering to the subject a therapeutically effective amount of (i) blocking antibodies to ECM components and/or (ii) inhibitors of collagen deposition or production.

4. The method of claim 1, wherein the blocking antibodies to ECM components bind to LAIR1/2; Cd29 (integrin R); CD49A; GPR561; integrin αV or integrin β3.

5. The method of claim 1, wherein the inhibitor of collagen deposition or production is losartan or 3,4-dihydroxybenzoic acid (DHB).

6. The method of claim 1, wherein the subject has breast, prostate, pancreatic, brain, hepatic, lung, kidney, skin, or colon cancer.

7. The method of claim 6, wherein the skin cancer is melanoma.

8. The method of claim 2, wherein the subject has a viral infection selected from a respiratory virus; a gastrointestinal virus; hepatitis A, B, C, D, and E viruses; arboviruses, arenaviruses, and filoviruses; and viruses that infect the skin or mucosal membranes.

9.-16. (canceled)

17. The method of claim 17, wherein the respiratory virus is selected from influenza viruses (A and B), H5N1 and H7N9 avian influenza A viruses, parainfluenza viruses 1 through 4, adenoviruses, respiratory syncytial virus A and B and human metapneumovirus, rhinoviruses, or coronaviruses.

18. The method of claim 17, wherein the gastrointestinal virus is selected from rotavirus, norovirus, astrovirus, adenovirus 40 and 41, coronavirus-like agents, or enteroviruses, optionally coxsackieviruses and echoviruses.

19. The method of claim 17, wherein the viruses that infect the skin or mucosal membranes are selected from herpes simplex viruses (HSV), papillomavirus, polyomavirus, and poxviruses.
